(19) United States
(12) Patent Application Publication
(10) Pub. No.: US 2022/0175740 A1
(43) Pub. Date: Jun. 9, 2022

(54) PHARMACEUTICAL COMPOSITIONS OF A SELECTIVE C-KIT KINASE INHIBITOR AND METHODS FOR MAKING AND USING SAME

(71) Applicant: Novartis AG, Basel (CH)

(72) Inventors: Michael JUHNKE, Basel (CH); Vijay Anand Sethuraman, Fremont, CA (US)

(21) Appl. No.: 17/455,733

(22) Filed: Nov. 19, 2021

Related U.S. Application Data
(60) Provisional application No. 63/115,685, filed on Nov. 19, 2020.

(51) Int. Cl. A61K 31/437; A61K 9/16

(52) U.S. Cl. CPC A61K 31/437; A61K 9/16

(57) ABSTRACT

The present disclosure relates generally to pharmaceutical compositions of N-(5-(5-((1R,2S)-2-fluorocyclopropyl)-1,2,4-oxadiazol-3-yl)-2-methylphenyl)imidazo[1,2-a]pyridine-3-carboxamide useful as a selective inhibitor of c-kit kinase and uses of the same in the treatment of c-kit kinase associated diseases.

### FIELD OF THE INVENTION

The present disclosure relates generally to pharmaceutical compositions of N-(5-(5-((1R,2S)-2-fluorocyclopropyl)-1,2,4-oxadiazol-3-yl)-2-methylphenyl)imidazo[1,2-a]pyridine-3-carboxamide useful as a selective inhibitor of c-kit kinase and uses of the same in the treatment of c-kit kinase associated diseases.

### BACKGROUND

N-(5-(5-((1R,2S)-2-fluorocyclopropyl)-1,2,4-oxadiazol-3-yl)-2-methylphenyl)imidazo[1,2-a]pyridine-3-carboxamide, first disclosed in WO 2013/033070 A1, is a selective inhibitor of c-kit kinase, useful for the depletion of mast cells and thus is useful for treating mast-cell associated diseases including asthma, allergic rhinitis, pulmonary arterial hypertension (PAH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, food allergy, type I diabetes and type II diabetes.

There remains a need in the art for novel compositions for delivering c-kit kinase inhibitors and methods for treating c-kit associated diseases using the same.

### SUMMARY OF THE INVENTION

It has now been found that pharmaceutical compositions of the present disclosure, and compositions thereof, are useful for administering a selective inhibitor of c-kit kinase to a patient in need thereof and exhibit desirable characteristics for the same. In general, the pharmaceutically acceptable compositions disclosed herein are useful for treating or lessening the severity of a variety of diseases or disorders as described in detail herein.

### BRIEF DESCRIPTION OF THE DRAWINGS

FIGS. 1A-1C depict XRPD patterns for Formulations 1A and 1B (FIG. 1A), 1C (FIG. 1B) and 1D (FIG. 1C), as compared to their respective starting materials.

FIGS. 2A-2D depict XRPD patterns for Formulations 1A (FIG. 2A), 1B (FIG. 2B), 2C (FIG. 2C) and 1D (FIG. 2D) after 1 week and 4 weeks stored at 25° C. and 60% relative humidity as compared to their respective starting materials and the formulation before the stability study.

FIG. 3 depicts an XRPD pattern for Formulation 2A as compared to the XRPD pattern for Compound 1 Form H^{A}.

FIG. 4 depicts XRPD patterns for Formulation 2A after 1 week and 4 weeks stored at either 4° C. or 25° C. and 60% relative humidity, with comparison XRPD patterns for Formulation 2A, as freshly prepared before the stability study, and Compound 1 Form H^{A}.

FIG. 5 depicts XRPD patterns for Formulation 2A after being frozen for 7 days, 3 days and 1 day, with comparison XRPD patterns for Formulation 2A, as freshly prepared before the freeze/thaw study, and Compound 1 Form H^{A}.

FIG. 6 is a photograph showing samples of Formulation 2A that had been frozen for 7 days, 3 days and 1 day. The photographs show a separation of layers in all three samples, most apparent in the 7 day sample, indicating a loss of homogeneity of the nanosuspension.

FIG. 7 depicts XRPD patterns for the scaled up preparation of Formulation 2A batches 3A-3E, as compared with the XRPD pattern for Compound 1 Form H^{A}.

FIG. 8 depicts XRPD patterns for scaled up Batch 3E after being stored for 3.5 months, with comparison XRPD patterns for Batch 3E, as freshly prepared before the stability study, and Compound 1 Form H^{A}.

FIG. 9 depicts XRPD patterns for the scaled up preparation of Formulation 2A batches 4A-4F, as compared with the XRPD pattern for Compound 1 Form H^{A}.

FIGS. 10A and 10B depict XRPD patterns for Formulations 5A (FIG. 10A) and 5B (FIG. 10B) as compared to their respective starting materials, Compound 1 Form H^{B} and Form D. Also included in FIG. 10B is the XRPD pattern for Compound 1 Form H^{B}, showing that Formulation 5B possesses certain characteristic peaks suggesting that the Compound 1 in the Formulation 5B nanosuspension has converted to the hydrate form.

FIGS. 11A and 11B depict XRPD patterns for Formulations 5A (FIG. 11A) and 5B (FIG. 11B) after 1 week and 4 weeks stored at 25° C. and 60% relative humidity as compared to their respective starting materials and the formulation before the stability study. Also included in FIG. 11B is the XRPD pattern for Compound 1 Form H^{B}, showing that Formulation 5B possesses certain characteristic peaks suggesting that the Compound 1 in the Formulation 5B nanosuspension has converted to the hydrate form.

### DETAILED DESCRIPTION OF THE INVENTION

The present disclosure is based at least in part on the identification of a compound that modulates c-kit kinase and methods of using the same to treat c-kit kinase associated diseases. Disclosed herein is compound 1, and pharmaceutical compositions thereof:

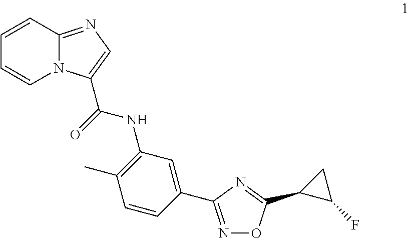 [SMILES: Cc1ccc(-c2noc([C@H]3C[C@@H]3F)n2)cc1NC(=O)c1cnc2ccccn12]

Compound 1, N-(5-(5-((1R,2S)-2-fluorocyclopropyl)-1,2,4-oxadiazol-3-yl)-2-methylphenyl)imidazo[1,2-a]pyridine-3-carboxamide, is active in a variety of assays and therapeutic models, acting as a selective inhibitor of c-kit kinase.

It would be desirable to provide pharmaceutically acceptable compositions comprising Compound 1 (e.g., as a freebase thereof or salt thereof) that imparts characteristics such as improved stability, improved oral bioavailability, and low toxicity risk. Accordingly, the present disclosure provides pharmaceutical compositions of Compound 1.

### Nanosuspension Compositions:

In one aspect, the present invention provides a pharmaceutical composition for oral administration of Compound 1 to a subject, wherein compound 1 is formulated as part of a nanosuspension. In some embodiments, the pharmaceutical composition of the present invention comprises, or consists essentially of.
(i) Compound 1;
(ii) a stabilizer;
(iii) a surfactant;
(iv) water; and
   optionally one or more of: a diluent, a preservative, a pH adjuster, a coloring agent, a sweetener and a flavorant. In some embodiments, the pharmaceutical composition comprises one, or more than one of any of the aforementioned components. For example, in some embodiments, the composition comprises two or more stabilizers.

In some embodiments, the present invention provides pharmaceutical compositions that allow for administration of high dosages of Compound 1 to a subject that unexpectedly yield low toxicity. The present invention relates to the unexpected discovery that nanosuspension formulations described herein are well tolerated at high dosages.

### A. Compound 1

As defined above, a pharmaceutical composition of the present invention is a nanosuspension comprising Compound 1. Compound 1 can be prepared according to example F110 of WO 2013/033070 A1, which is incorporated by reference herein, as summarized in the Scheme 1 provided below:

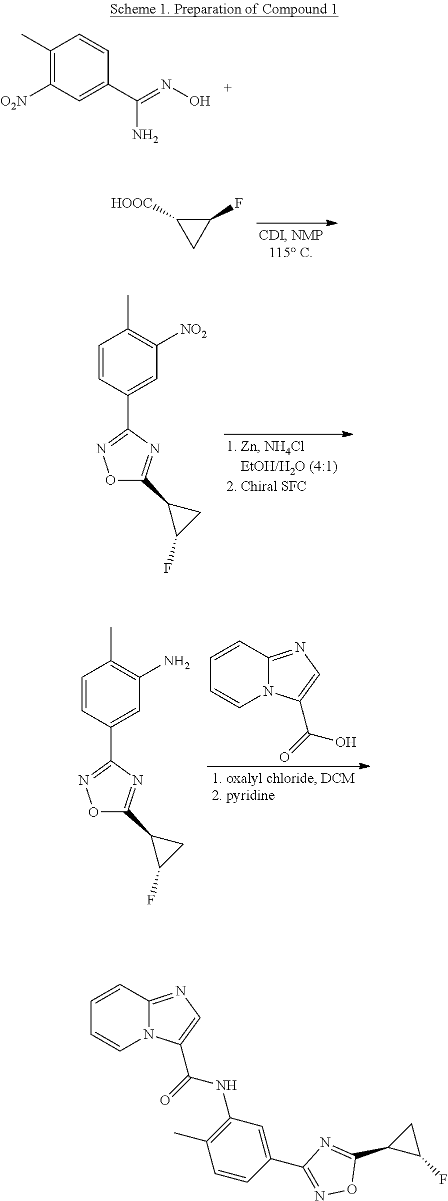

In some embodiments, the pharmaceutical composition is a nanosuspension comprising nanoparticles of Compound 1 suspended in an aqueous solution. In some embodiments, the nanoparticles of Compound 1 suspended in the aqueous solution comprise a crystalline solid form of Compound 1. In some embodiments, the nanoparticles of Compound 1 comprise a crystalline free base solid form of Compound 1. In some embodiments, the nanoparticles of Compound 1 comprise a crystalline salt solid form of Compound 1.

In some embodiments, the crystalline solid form of Compound 1 is an anhydrate form. In some embodiments, the crystalline solid form of Compound 1 is a hydrate form. In some embodiments, the crystalline solid form of Compound 1 is a monohydrate. In some embodiments, the crystalline solid form of Compound 1 is a hemihydrate. In some embodiments, the crystalline solid form of Compound 1 is a dihydrate.

In some embodiments, the nanoparticles of Compound 1 comprise a crystalline solid form of Compound 1 disclosed in PCT/CN2020/090060, which is incorporated by reference herein.

In some embodiments, the nanoparticles of Compound 1 comprise free base Form A of Compound 1. In some embodiments, the nanoparticles of Compound 1 consist of free base Form A of Compound 1. In some embodiments, Form A of Compound 1 is a form having at least 1, 2, 3, 4 or 5 X-ray powder diffraction spectral peak(s) selected from the peaks listed in Table 1A below.

TABLE 1A
XRPD Peak Positions for
Form A of Compound 1

| Position (°2θ) | Intensity % |
|---|---|
| 5.0 | 11.2 |
| 8.8 | 7.6 |
| 9.8 | 29.3 |
| 10.1 | 17.5 |
| 11.4 | 3.2 |
| 13.2 | 59.7 |
| 15.2 | 100 |
| 17.1 | 17.3 |
| 17.4 | 19.4 |
| 17.6 | 14.4 |
| 18.5 | 9.3 |
| 19.7 | 68.7 |

In this and all subsequent tables, the position (°2θ) is within ± 0.2.

In some embodiments, Form A of compound 1 is characterized in that it has one or more peaks in its X-ray powder diffraction pattern selected from those at about 13.2, about 15.2, and about 19.7 degrees 2-theta. In some embodiments, Form A of compound 1 is characterized in that it has two or more peaks in its X-ray powder diffraction pattern selected from those at about 13.2, about 15.2, and about 19.7 degrees 2-theta. In some embodiments, Form A of compound 1 is characterized in that it has three peaks in its X-ray powder diffraction pattern at about 13.2, about 15.2, and about 19.7 degrees 2-theta. In some embodiments, Form A of Compound 1 is characterized by a DSC thermogram having an endothermic event at about 175° C. In some embodiments, Form A of Compound 1 is characterized by a TGA curve showing insignificant mass loss up to a temperature of about 180° C.

Form A can be isolated by the following procedure: Dissolve about 2.0 g of amorphous compound 1 in 40 mL of isopropanol at 70° C. and mechanically stir for 3 hours. Cool the solution to room temperature and continue stirring overnight. A precipitate forms overnight and is filtered and washed with isopropanol and dried overnight at 60° C. under vacuum to yield Form A of Compound 1.

In some embodiments, the nanoparticles of Compound 1 are substantially pure. In some embodiments, the nanoparticles of Compound 1 comprise free base Form A and are substantially free of amorphous Compound 1 and other crystalline forms of Compound 1. As used herein, the term “substantially free” means that the compound contains no significant amount of amorphous Compound 1 or other crystalline forms. In certain embodiments, the nanoparticles comprise at least about 95% by weight of crystalline Compound 1 Form A. In still other embodiments of the disclosure, the nanoparticles comprise at least about 99% by weight of crystalline Compound 1 Form A.

In some embodiments, the nanoparticles of Compound 1 comprise free base Form H^{A} of Compound 1. In some embodiments, the nanoparticles of Compound 1 consist of free base Form H^{A} of Compound 1. In some embodiments, Form H^{A} of Compound 1 is a form having at least 1, 2, 3, 4 or 5 X-ray powder diffraction spectral peak(s) selected from the peaks listed in Table 1B below.

TABLE 1B
XRPD Peak Positions for
Form H^{A} of Compound 1

| Position (°2θ) | Intensity % |
|---|---|
| 6.4 | 12.4 |
| 8.0 | 4.0 |
| 10.1 | 2.2 |
| 10.7 | 10.4 |
| 12.8 | 100 |
| 13.6 | 37.0 |
| 16.3 | 3.3 |
| 16.8 | 8.0 |
| 18.4 | 7.0 |
| 19.3 | 27.1 |
| 19.9 | 11.3 |
| 21.6 | 2.9 |
| 25.6 | 8.7 |
| 26.9 | 3.5 |
| 32.6 | 3.2 |

In this and all subsequent tables, the position (°2θ) is within ± 0.2.

In some embodiments, Form H^{A} of compound 1 is characterized in that it has one or more peaks in its X-ray powder diffraction pattern selected from those at about 12.8, about 13.6, and about 19.3 degrees 2-theta. In some embodiments, Form H^{A} of compound 1 is characterized in that it has two or more peaks in its X-ray powder diffraction pattern selected from those at about 12.8, about 13.6, and about 19.3 degrees 2-theta. In some embodiments, Form H^{A} of compound 1 is characterized in that it has three peaks in its X-ray powder diffraction pattern at about 12.8, about 13.6, and about 19.3 degrees 2-theta. In some embodiments, Form H^{A} of Compound 1 is characterized by a DSC thermogram having one or more endothermic events at temperatures selected from about 87° C., about 125° C., about 165° C. and about 175° C. In some embodiments, Form H^{A} of Compound 1 is characterized by a TGA curve showing about a 5% mass loss up to a temperature of about 112° C.

Form H^{A} can be isolated by the following procedure: Add about 200 mg of Form A of Compound 1 to 3.0 mL of MeOH/H₂O (1:1, v/v) and stir at 1000 rpm at room temperature for 5 days. Centrifuge the suspension, collect the solids and dry under vacuum to yield Form H^{A} of Compound 1.

In some embodiments, the nanoparticles of Compound 1 are substantially pure. In some embodiments, the nanoparticles of Compound 1 comprise free base Form H^{A} and are substantially free of amorphous Compound 1 and other crystalline forms of Compound 1. In certain embodiments, the nanoparticles comprise at least about 95% by weight of crystalline Compound 1 Form H^{A}. In still other embodiments of the disclosure, the nanoparticles comprise at least about 99% by weight of crystalline Compound 1 Form H^{A}.

In some embodiments, the nanoparticles of Compound 1 comprise free base Form H^{B} of Compound 1. In some embodiments, the nanoparticles of Compound 1 consist of free base Form H^{B} of Compound 1. In some embodiments, Form H^{B} of Compound 1 is a form having at least 1, 2, 3, 4 or 5 X-ray powder diffraction spectral peak(s) selected from the peaks listed in Table 1C below.

TABLE 1C
XRPD Peak Positions for
Form H^{B} of Compound 1

| Position (°2θ) | Intensity % |
|---|---|
| 6.7 | 32.2 |
| 10.1 | 27.0 |
| 10.7 | 24.1 |
| 11.2 | 13.3 |
| 13.6 | 100 |
| 16.5 | 15.4 |
| 18.0 | 73.3 |
| 19.1 | 56.6 |
| 20.2 | 24.0 |
| 23.5 | 35.1 |
| 23.8 | 45.8 |
| 25.0 | 42.4 |
| 26.4 | 54.7 |
| 28.7 | 19.3 |
| 29.7 | 34.5 |

In this and all subsequent tables, the position (°2θ) is within ± 0.2.

In some embodiments, Form H^{B} of compound 1 is characterized in that it has one or more peaks in its X-ray powder diffraction pattern selected from those at about 13.6, about 18.0, and about 26.4 degrees 2-theta. In some embodiments, Form H^{B} of compound 1 is characterized in that it has two or more peaks in its X-ray powder diffraction pattern selected from those at about 13.6, about 18.0, and about 26.4 degrees 2-theta. In some embodiments, Form H^{B} of compound 1 is characterized in that it has three peaks in its X-ray powder diffraction pattern at about 13.6, about 18.0, and about 26.4 degrees 2-theta. In some embodiments, Form H^{B} of Compound 1 is characterized by a DSC thermogram having one or more endothermic events at temperatures selected from about 110° C., about 125° C., about 165° C. and about 173° C. In some embodiments, Form H^{B} of Compound 1 is characterized by a TGA curve showing about a 5.4% mass loss up to a temperature of about 150° C.

Form H^{B} can be isolated by the following procedure: Add about Form A of Compound 1 to a vial of water and leave at room temperature for two weeks. Centrifuge the suspension, collect the solids and dry under vacuum to yield Form H^{B} of Compound 1.

In some embodiments, the nanoparticles of Compound 1 are substantially pure. In some embodiments, the nanoparticles of Compound 1 comprise free base Form H^{B} and are substantially free of amorphous Compound 1 and other crystalline forms of Compound 1. In certain embodiments, the nanoparticles comprise at least about 95% by weight of crystalline Compound 1 Form H^{B}. In still other embodiments of the disclosure, the nanoparticles comprise at least about 99% by weight of crystalline Compound 1 Form H^{B}.

In some embodiments, the nanoparticles of Compound 1 comprise free base Form D of Compound 1. In some embodiments, the nanoparticles of Compound 1 consist of free base Form D of Compound 1. In some embodiments, Form D of Compound 1 is a form having at least 1, 2, 3, 4 or 5 X-ray powder diffraction spectral peak(s) selected from the peaks listed in Table 1D below.

TABLE 1D
XRPD Peak Positions for
Form D of Compound 1

| Position (°2θ) | Intensity % |
|---|---|
| 3.1 | 14.1 |
| 5.1 | 8.8 |
| 8.9 | 34.7 |
| 9.9 | 80.7 |
| 10.2 | 9.5 |
| 11.4 | 4.8 |
| 13.3 | 71.7 |
| 15.3 | 46.3 |
| 17.2 | 90.7 |
| 17.7 | 61.8 |
| 18.6 | 28.2 |
| 19.8 | 100.0 |
| 20.4 | 10.6 |
| 21.3 | 2.5 |
| 22.1 | 17.2 |
| 22.9 | 10.8 |
| 24.6 | 3.9 |
| 26.1 | 28.0 |
| 26.9 | 4.2 |
| 27.6 | 5.1 |
| 27.9 | 2.5 |
| 29.9 | 2.4 |
| 31.4 | 2.2 |
| 32.0 | 0.8 |
| 33.0 | 1.5 |
| 34.3 | 2.0 |
| 34.7 | 2.0 |

In this and all subsequent tables, the position (°2θ) is within ± 0.2.

In some embodiments, Form D of compound 1 is characterized in that it has one or more peaks in its X-ray powder diffraction pattern selected from those at about 8.9, about 9.9, about 13.3, about 15.3, about 17.2, about 17.7, about 19.8, and about 26.1 degrees 2-theta. In some embodiments, Form D of compound 1 is characterized in that it has two or more peaks in its X-ray powder diffraction pattern selected from those at about 8.9, about 9.9, about 13.3, about 15.3, about 17.2, about 17.7, about 19.8, and about 26.1 degrees 2-theta. In some embodiments, Form D of compound 1 is characterized in that it has three or more peaks in its X-ray powder diffraction pattern selected from those at about 8.9, about 9.9, about 13.3, about 15.3, about 17.2, about 17.7, about 19.8, and about 26.1 degrees 2-theta. In some embodiments, Form D of compound 1 is characterized in that it has four or more peaks in its X-ray powder diffraction pattern selected from those at about 8.9, about 9.9, about 13.3, about 15.3, about 17.2, about 17.7, about 19.8, and about 26.1 degrees 2-theta. In some embodiments, Form D of compound 1 is characterized in that it has five or more peaks in its X-ray powder diffraction pattern selected from those at about 8.9, about 9.9, about 13.3, about 15.3, about 17.2, about 17.7, about 19.8, and about 26.1 degrees 2-theta. In some embodiments, Form D of compound 1 is characterized in that it has six or more peaks in its X-ray powder diffraction pattern selected from those at about 8.9, about 9.9, about 13.3, about 15.3, about 17.2, about 17.7, about 19.8, and about 26.1 degrees 2-theta. In some embodiments, Form D of compound 1 is characterized in that it has seven or more peaks in its X-ray powder diffraction pattern selected from those at about 8.9, about 9.9, about 13.3, about 15.3, about 17.2, about 17.7, about 19.8, and about 26.1 degrees 2-theta. In some embodiments, Form D of compound 1 is characterized in that it has eight peaks in its X-ray powder diffraction pattern at about 8.9, about 9.9, about 13.3, about 15.3, about 17.2, about 17.7, about 19.8, and about 26.1 degrees 2-theta. In some embodiments, Form D of Compound 1 is characterized by a DSC thermogram having an endothermic event at about 175° C. In some embodiments, Form D of Compound 1 is characterized by a TGA curve showing minimal mass loss up to a temperature of about 250° C.

Form D can be isolated by the following procedure: Add about 200 mg of Form A of Compound 1 to 3.0 mL of isopropanol and stir at 1000 rpm at room temperature for 5 days. Centrifuge the suspension, collect the solids and dry under vacuum to yield Form D of Compound 1.

In some embodiments, the nanoparticles of Compound 1 are substantially pure. In some embodiments, the nanoparticles of Compound 1 comprise free base Form D and are substantially free of amorphous Compound 1 and other crystalline forms of Compound 1. In certain embodiments, the nanoparticles comprise at least about 95% by weight of crystalline Compound 1 Form D. In still other embodiments of the disclosure, the nanoparticles comprise at least about 99% by weight of crystalline Compound 1 Form D.

In some embodiments, the nanoparticles of Compound 1 comprise HCl salt Form I of Compound 1. In some embodiments, the nanoparticles of Compound 1 consist of HCl salt Form I of Compound 1. Form I of Compound 1 is an anhydrate form having a molar ratio of HCl to Compound 1 of about 1:1. In some embodiments, Form I of Compound 1 is a form having at least 1, 2, 3, 4 or 5 X-ray powder diffraction spectral peak(s) selected from the peaks listed in Table 1E below.

TABLE 1E
XRPD Peak Positions
for Form I of Compound 1

| Position (°2θ) | Intensity % |
|---|---|
| 6.0 | 22.4 |
| 10.8 | 31.2 |
| 11.3 | 6.2 |
| 11.9 | 15.0 |
| 12.8 | 28.5 |
| 15.0 | 12.2 |
| 16.4 | 2.1 |
| 17.4 | 6.6 |
| 17.9 | 75.2 |
| 18.1 | 25.6 |
| 19.3 | 18.1 |
| 20.2 | 18.1 |
| 21.7 | 11.6 |
| 22.5 | 4.0 |
| 23.5 | 4.7 |
| 24.0 | 7.0 |
| 25.6 | 76.7 |
| 26.5 | 100 |
| 27.7 | 3.8 |
| 29.0 | 10.3 |
| 30.0 | 2.3 |
| 31.5 | 2.6 |
| 32.9 | 2.6 |
| 34.4 | 1.5 |
| 38.0 | 1.8 |
| 39.2 | 1.0 |

In this and all subsequent tables, the position (°2θ) is within ± 0.2.

In some embodiments, Form I of compound 1 is characterized in that it has one or more peaks in its X-ray powder diffraction pattern selected from those at about 17.9, about 25.6 and about 26.5 degrees 2-theta. In some embodiments, Form I of compound 1 is characterized in that it has two or more peaks in its X-ray powder diffraction pattern selected from those about 17.9, about 25.6 and about 26.5 degrees 2-theta. In some embodiments, Form I of compound 1 is characterized in that it has three peaks in its X-ray powder diffraction pattern at about 17.9, about 25.6 and about 26.5 degrees 2-theta. In some embodiments, Form I of Compound 1 is characterized by a DSC thermogram having an endothermic event at about 258.6° C. In some embodiments, Form I of Compound 1 is characterized by a TGA curve showing minimal mass loss up to a temperature of about 150° C.

Form I can be isolated by the following procedure: Add about 2 g of Compound 1 free base to 20 mL of isopropanol in a first vial and stir to obtain a suspension. Add about 250 mg HCl (36 wt %) to 20 mL of isopropanol in a second vial to obtain a solution. Add the contents of the second vial to the first vial and stir (500 rpm) at room temperature for 3 days. Filter the contents of the vial and dry the solid under vacuum at room temperature overnight yield Form I of Compound 1.

In some embodiments, the nanoparticles of Compound 1 hydrochloride salt are substantially pure. In some embodiments, the nanoparticles of Compound 1 comprise free base HCl salt Form I and are substantially free of amorphous Compound 1 and other crystalline forms of Compound 1. In certain embodiments, the nanoparticles comprise at least about 95% by weight of crystalline Compound 1 HCl salt Form I. In still other embodiments of the disclosure, the nanoparticles comprise at least about 99% by weight of crystalline Compound 1 HCl salt Form I.

In some embodiments, the nanoparticles of Compound 1 have a median particle size (D₅₀) of about 75 nm to about 250 nm with a span ([D₉₀-D₁₀]/D₅₀) less than about 2. In some embodiments, the nanoparticles of Compound 1 have a median particle size of about 100 nm to about 250 nm, with a span less than about 1.75. In some embodiments, the nanoparticles of Compound 1 have a median particle size of about 100 nm to about 200 nm, with a span less than about 1.75. In some embodiments, the nanoparticles of Compound 1 have a median particle size of about 100 nm to about 250 nm, with a span less than about 1.5. In some embodiments, the nanoparticles of Compound 1 have a median particle size of about 100 nm to about 200 nm, with a span less than about 1.5. In some embodiments, the nanoparticles of Compound 1 have a median particle size of about 100 nm to about 150 nm, with a span less than about 1.5. In some embodiments, the nanoparticles of Compound 1 have a median particle size of about 75 nm to about 250 nm. In some embodiments, the nanoparticles of Compound 1 have a median particle size of about 75 nm to about 200 nm. In some embodiments, the nanoparticles of Compound 1 have a median particle size of about 75 nm to about 175 nm. In some embodiments, the nanoparticles of Compound 1 have a median particle size of about 75 nm to about 150 nm. In some embodiments, the nanoparticles of Compound 1 have a median particle size of about 100 nm to about 250 nm. In some embodiments, the nanoparticles of Compound 1 have a median particle size of about 100 nm to about 200 nm. In some embodiments, the nanoparticles of Compound 1 have a median particle size of about 100 nm to about 175 nm. In some embodiments, the nanoparticles of Compound 1 have a median particle size of about 100 nm to about 150 nm.

In some embodiments, the nanoparticles of Compound 1 have a median particle size (D₅₀) of about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, or about 250 nm. In some embodiments, the nanoparticles of Compound 1 have a particle size distribution span ([D₉₀-D₁₀]/D₅₀) less than about 2, less than about 1.9, less than about 0.8, less than about 1.75, less than about 1.7, less than about 1.6, less than about 1.5, less than about 1.4, less than about 1.3, less than about 1.2, less than about 1.1, less than about 1, less than about 0.9, less than about 0.8, less than 0.7, less than about 0.6 or less than about 0.5.

In some embodiments, Compound 1 is present in the pharmaceutical composition in an amount from about 1 wt % to about 20 wt %. In some embodiments, Compound 1 is present in the pharmaceutical composition in an amount from about 5 wt % to about 15 wt %. In some embodiments, Compound 1 is present in the pharmaceutical composition in an amount from about 8 wt % to about 12 wt %. In some embodiments, Compound 1 is present in the pharmaceutical composition in an amount of about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt %. In some embodiments, Compound 1 is present in the pharmaceutical composition in an amount of about 10 wt %.

In some embodiments, the pharmaceutical composition comprises nanoparticles of Compound 1 in the form of free base solid form H^{A}, wherein the nanoparticles have a median particle size of about 75 nm to about 250 nm, with a particle size distribution span less than about 2, and wherein Compound 1 is present in the pharmaceutical composition in an amount of about 10 wt %. In some embodiments, the pharmaceutical composition comprises nanoparticles of Compound 1 in the form of free base solid form H^{A}, wherein the nanoparticles have a median particle size of about 100 nm to about 200 nm, with a particle size distribution span less than about 1.5, and wherein Compound 1 is present in the pharmaceutical composition in an amount of about 10 wt %.

### B. Stabilizers

As defined above, a pharmaceutical composition of the present invention is a nanosuspension comprising a stabilizer.

In some embodiments, the stabilizer comprises an organic polymer. In some embodiments, the stabilizer comprises an organic polymer comprising cellulose or a derivative thereof. In some embodiments, the stabilizer comprises hydroxypropylcellulose (HPC) or hydroxypropyl methylcellulose (HPMC). In some embodiments, the stabilizer comprises an organic polymer. In some embodiments, the organic polymer comprises polypropylene oxide, polyethylene oxide or a combination thereof. In some embodiments, the stabilizer comprises an organic polymer comprising polyvinylpyrrolidone, or a derivative thereof. In some embodiments, the stabilizer comprises polyvinylpyrrolidone, or a polyvinylpyrrolidone copolymer. In some embodiments, the stabilizer comprises a polyvinylpyrollidone-vinyl acetate (PVP/VA) copolymer. In some embodiments, the stabilizer comprises a polyvinylpyrollidone-vinyl acetate (PVP/VA) copolymer having a weight-average molecular weight of about 45,000 to about 70,000. In some embodiments, the stabilizer comprises hydroxypropyl methylcellulose acetate succinate.

In some embodiments, the stabilizer comprises one or more commercial stabilizers selected from EUDRAGIT EPO, PVP K-30 polymer (ASHLAND™), KOLLIDON® VA 64 (BASF®), Plasdone K-29/32 (ASHLAND™), KLUCEL™ HPC (ASHLAND™), and HPMC PHARMACOAT® 603.

In some embodiments, the stabilizer comprises KOLLIDON® VA 64 (BASF®), a polyvinylpyrolidone-vinyl acetate (PVP/VA) copolymer, also referred to as copolyvidone or copovidone. KOLLIDON® VA 64 (BASF®) has a weight-average molecular weight of 45,000-70,000 g/mol, or a K-value of 45 to 70, and comprises 3 parts N-vinylpyrrolidone to 2 parts vinyl acetate

In some embodiments, the stabilizer is any stabilizer commonly utilized in the formulation of pharmaceutical compositions for oral administration.

In some embodiments, the stabilizer is present in the pharmaceutical composition in an amount from about 1 wt % to about 10 wt %. In some embodiments, the stabilizer is present in the pharmaceutical composition in an amount from about 2 wt % to about 5 wt %. In some embodiments, the stabilizer is present in the pharmaceutical composition in an amount of about 1 wt %, 2 wt %, about 3 wt %, about 4 wt %, about 5 wt %, about 6 wt %, about 7 wt %, about 8 wt %, about 9 wt %, or about 10 wt %. In some embodiments, the stabilizer is present in the pharmaceutical composition in an amount of about 3 wt %.

In some embodiments, the pharmaceutical composition comprises a stabilizer which is KOLLIDON® VA 64 in an amount of about 3 wt %.

### C. Surfactant

As defined above, a pharmaceutical composition of the present invention is a nanosuspension comprising a surfactant.

In some embodiments, the pharmaceutical composition comprises a surfactant selected from polyoxylethylene stearate, sorbitan stearate, sorbitan sesquioleate, sorbitan monooleate, Polysorbate 20, Polysorbate 80, sodium dodecyl sulfate (SDS; alternatively referred to as sodium lauryl sulfate, abbreviated as SLS) and bis(2-ethylhexyl) sulfosuccinate, also known as dioctyl sulfosuccinate (DOSS). In some embodiments, the surfactant is polysorbate 80. In some embodiments, the surfactant is SDS. In some embodiments, the surfactant is DOSS. In some embodiments, the surfactant is a polyoxyethylene alkyl ether, an alkylphenylpolyoxyethylene ether, or a polysorbate.

In some embodiments, the surfactant is any surfactant commonly utilized in the formulation of pharmaceutical compositions for oral administration.

In some embodiments, the surfactant is present in the pharmaceutical composition in an amount from about 0.01 wt % to about 0.20 wt %. In some embodiments, the surfactant is present in the pharmaceutical composition in an amount from about 0.05 wt % to about 0.15 wt %. In some embodiments, the surfactant is present in the pharmaceutical composition in an amount from about 0.08 wt % to about 0.12 wt %. In some embodiments, the surfactant is present in the pharmaceutical composition in an amount of about 0.01 wt %, 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.11 wt %, about 0.12 wt %, about 0.13 wt %, about 0.14 wt %, about 0.15 wt %, about 0.16 wt %, about 0.17 wt %, about 0.18 wt %, about 0.19 wt %, or about 0.20 wt %. In some embodiments, the surfactant is present in the pharmaceutical composition in an amount of about 0.1 wt %.

In some embodiments, the pharmaceutical composition comprises a surfactant which is SDS in an amount of about 0.1 wt %.

### D. Additional Ingredients

As defined above, a pharmaceutical composition of the present invention is a nanosuspension optionally comprising one or more additional ingredients, selected from, but not necessarily limited to, a diluent, a preservative, a pH adjuster, a coloring agent, a sweetener and a flavorant.

In some embodiments, the pharmaceutical composition optionally comprises a diluent, bulking agent or filler. In some embodiments, the diluent is selected from sorbitol, isomalt, mannitol, starch, cellulose, or combinations thereof.

In some embodiments, the pharmaceutical composition optionally comprises a preservative. Suitable preservatives include, but are not limited to, antimicrobial agents and/or antioxidants. Suitable antimicrobial agents can include, but are not limited to, benzoates, benzyl alcohol, sodium benzoate, sorbates, propionates, and nitrites. Suitable antioxidants can include, but are not limited to, vitamin C, butylated hydroxytoluene (BHT), sulphites, and vitamin E. In some embodiments, the preservative is selected from vitamin A, vitamin C, vitamin E, vitamin E TPGS, retinyl palmitate, selenium, cysteine, methionine, citric acid, sodium citrate, methyl paraben, propyl paraben, disodium edetate, butylated hydroxy toluene, riboflavin, ascorbic acid or combinations thereof.

In some embodiments, the pharmaceutical composition optionally comprises a pH adjuster. In some embodiments, the pH adjuster is a pharmaceutically acceptable acid or base. In some embodiments, the pH adjuster is a buffer. In some embodiments, the pH adjuster is a citric buffer, a malate buffer, a maleate buffer, or a tartrate buffer. In some embodiments, the pH adjuster is ascorbic acid, glutathione, cysteine, methionine, citric acid, EDTA, malic acid, sodium malate, tartaric acid, disodium tartrate, or any combinations thereof. In some embodiments, the pH adjuster is selected from vitamin A, vitamin C, vitamin E, vitamin E TPGS, retinyl palmitate, selenium, cysteine, methionine, citric acid, sodium citrate, methyl paraben, propyl paraben, disodium edetate, butylated hydroxy toluene, riboflavin, ascorbic acid or combinations thereof.

In some embodiments, the pharmaceutical composition optionally comprises one or more sweeteners and flavorants to improve the palatability of the composition. In some embodiments, the pharmaceutical composition comprises a flavorant such as, but not limited to, a vanilla flavoring or a strawberry flavoring. In some embodiments, the pharmaceutical composition comprises a sweetener such as, but not limited to, sucralose, aspartame, sodium saccharin or calcium saccharin.

### E. Formulation

As described above, in some embodiments, the pharmaceutical composition is a nanosuspension comprising, or consisting essentially of:
(i) Compound 1;
(ii) a stabilizer;
(iii) a surfactant;
(iv) water; and
   optionally one or more of: a diluent, a preservative, a pH adjuster, a coloring agent, a sweetener and a flavorant.

In some embodiments, the pharmaceutical composition comprises, or consists essentially of:
(i) Compound 1 in an amount of about 5 wt % to about 15 wt %;
(ii) a stabilizer in an amount of about 1 wt % to about 10 wt %;
(iii) a surfactant in an amount of about 0.05 wt % to about 0.15 wt %;
(iv) water; and
   optionally one or more of: a diluent, a preservative, a pH adjuster, a coloring agent, a sweetener and a flavorant.

In some embodiments, the pharmaceutical composition comprises, or consists essentially of:
(i) Compound 1 in an amount of about 5 wt % to about 15 wt %;
(ii) a stabilizer in an amount of about 1 wt % to about 10 wt %;
(iii) a surfactant in an amount of about 0.05 wt % to about 0.15 wt %; and
(iv) water.

In some embodiments, the pharmaceutical composition comprises, or consists essentially of:
(i) Compound 1 in an amount of about 8 wt % to about 12 wt %;
(ii) a stabilizer in an amount of about 2 wt % to about 5 wt %;
(iii) a surfactant in an amount of about 0.08 wt % to about 0.12 wt %; and
(iv) water, wherein the water makes up the mass balance of the composition.

In some embodiments, the pharmaceutical composition comprises, or consists essentially of:
(i) Compound 1 in an amount of about 10 wt %;
(ii) a stabilizer in an amount of about 3 wt %;
(iii) a surfactant in an amount of about 0.1 wt %; and
(iv) water in an amount of about 86.9 wt %.

In some embodiments, the pharmaceutical composition comprises, or consists essentially of:
(i) Compound 1 in the form of crystalline free base Form H^{A};
(ii) a polyvinylpyrolidone-polyvinyl acetate (PVP/VA) copolymer;
(iii) SDS; and
(iv) water.

In some embodiments, the pharmaceutical composition comprises, or consists essentially of:
(i) Compound 1 in the form of crystalline free base Form H^{A};
(ii) PVP/VA;
(iii) SDS; and
(iv) water.

In some embodiments, the pharmaceutical composition comprises, or consists essentially of:
(i) Compound 1 in the form of crystalline free base Form H^{A} nanoparticles having a median particle size of about 75 nm to about 250 nm, with a particle size distribution span less than about 2;
(ii) PVP/VA;
(iii) SDS; and
(iv) water.

In some embodiments, the pharmaceutical composition comprises, or consists essentially of:
(i) Compound 1 in the form of crystalline free base Form H^{A} nanoparticles having a median particle size of about 75 nm to about 250 nm, with a particle size distribution span less than about 2, in an amount of about 8 wt % to about 12 wt %;
(ii) PVP/VA in an amount of about 2 wt % to about 5 wt %;
(iii) SDS in an amount of about 0.08 wt % to about 0.12 wt %; and
(iv) water, wherein the water makes up the mass balance of the composition.

In some embodiments, the pharmaceutical composition comprises, or consists essentially of:
(i) Compound 1 in the form of crystalline free base Form H^{A} nanoparticles having a median particle size of about 75 nm to about 250 nm, with a particle size distribution span less than about 2, in an amount of about 10 wt %;
(ii) PVP/VA in an amount of about 3 wt %;
(iii) SDS in an amount of about 0.1 wt %; and
(iv) water, in an amount of about 86.9 wt %.

In some embodiments, the pharmaceutical composition comprises, or consists essentially of:
(i) Compound 1 in the form of crystalline free base Form H^{A} nanoparticles having a median particle size of about 100 nm to about 200 nm, with a particle size distribution span less than about 1.5;
(ii) PVP/VA;
(iii) SDS; and
(iv) water.

In some embodiments, the pharmaceutical composition comprises, or consists essentially of:
(i) Compound 1 in the form of crystalline free base Form H^{A} nanoparticles having a median particle size of about 100 nm to about 200 nm, with a particle size distribution span less than about 1.5, in an amount of about 8 wt % to about 12 wt %;
(ii) PVP/VA in an amount of about 2 wt % to about 5 wt %;
(iii) SDS in an amount of about 0.08 wt % to about 0.12 wt %; and
(iv) water, wherein the water makes up the mass balance of the composition.

In some embodiments, the pharmaceutical composition comprises, or consists essentially of:
(i) Compound 1 in the form of crystalline free base Form H^{A} nanoparticles having a median particle size of about 100 nm to about 200 nm, with a particle size distribution span less than about 1.5, in an amount of about 10 wt %;
(ii) PVP/VA in an amount of about 3 wt %;
(iii) SDS in an amount of about 0.1 wt %; and
(iv) water, in an amount of about 86.9 wt %.

In some embodiments, any of the pharmaceutical compositions described above optionally further comprise one or more of: a diluent, a preservative, a pH adjuster, a coloring agent, a sweetener and a flavorant.

In some embodiments, any of the pharmaceutical compositions described above comprise a polyvinylpyrolidone-polyvinyl acetate (PVP/VA) copolymer having a weight-average molecular weight of about 45,000 to about 70,000.

### Uses of Compounds and Pharmaceutically Acceptable Compositions

As described generally above, compound 1, and pharmaceutically acceptable solid compositions thereof described herein, are inhibitors of c-kit kinase. The c-kit kinase inhibiting compounds of the present disclosure can, in some embodiments, find use in inhibiting activity of a target c-kit kinase in vitro or in vivo. Aspects of the subject methods include contacting a sample comprising an effective amount of a c-kit kinase inhibiting compound (e.g., as described herein) to determine whether the desired activity exists.

In one aspect, the present disclosure provides methods for treating a c-kit kinase mediated disease or disorder in a subject in need thereof. In some embodiments, the method comprises administering to the subject in need thereof a therapeutically effective amount of a pharmaceutical composition disclosed herein, i.e., a pharmaceutical composition comprising Compound 1. In some embodiments, the disease or disorder is a mast-cell associated disease, a respiratory disease, an inflammatory disorder, an autoimmune disorder, a metabolic disease, a fibrosis disease, or a dermatological disease. In some embodiments, the disease or disorder is asthma, allergic rhinitis, pulmonary arterial hypertension (PAH), primary pulmonary hypertension (PPH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, food allergy, type I diabetes or type II diabetes. In some embodiments, the administration is oral administration.

In another aspect, the present disclosure provides a pharmaceutical composition as disclosed herein, i.e., a pharmaceutical composition comprising Compound 1, for use in treating a c-kit kinase mediated disease or disorder in a subject in need thereof. In yet another aspect, the present disclosure provides a pharmaceutical composition as disclosed herein, i.e., a pharmaceutical composition comprising Compound 1, for the manufacture of a medicament for treating a c-kit kinase mediated disease or disorder in a subject in need thereof. In some embodiments, the disease or disorder is a mast-cell associated disease, a respiratory disease, an inflammatory disorder, an autoimmune disorder, a metabolic disease, a fibrosis disease, or a dermatological disease. In some embodiments, the disease or disorder is asthma, allergic rhinitis, pulmonary arterial hypertension (PAH), primary pulmonary hypertension (PPH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, food allergy, type I diabetes or type II diabetes.

As used herein, the terms “combination,” “combined,” and related terms refer to the simultaneous or sequential administration of therapeutic agents in accordance with this disclosure. For example, a described pharmaceutical composition may be administered with another therapeutic agent simultaneously or sequentially in separate unit dosage forms or together in a single unit dosage form.

When the pharmaceutical composition of this disclosure are administered in combination therapies with other agents, they may be administered sequentially or concurrently to the patient. Alternatively, pharmaceutical or prophylactic compositions according to this disclosure comprise a combination of Compound 1, or any other compound described herein, and another therapeutic or prophylactic agent. Additional therapeutic agents that are normally administered to treat a particular disease or condition may be referred to as “agents appropriate for the disease, or condition, being treated.”

In some embodiments, the subject method includes administering a therapeutically effective amount of one or more additional active agents. By combination therapy is meant that a c-kit inhibiting pharmaceutical composition can be used in a combination with another therapeutic agent to treat a single disease or condition. In particular embodiments, a pharmaceutical composition of the present disclosure is administered concurrently with the administration of another therapeutic agent.

The subject pharmaceutical composition can be administered in combination with other therapeutic agents in a variety of therapeutic applications. Therapeutic applications of interest for combination therapy include those applications in which activity of a target c-kit kinase is the cause or a compounding factor in disease progression. As such, the subject pharmaceutical composition find use in combination therapies in which the inhibition of a target c-kit kinase in the subject is desired.

The term “treatment” is used interchangeably herein with the term “therapeutic method” and refers to both 1) therapeutic treatments or measures that cure, slow down, lessen symptoms of, and/or halt progression of a diagnosed pathologic conditions, disease or disorder, and 2) and prophylactic/preventative measures. Those in need of treatment may include individuals already having a particular medical disease or disorder as well as those who may ultimately acquire the disorder (i.e., those at risk or needing preventive measures).

The term “subject” as used herein refers to any individual or patient to which the subject methods are performed. Generally, the subject is human, although as will be appreciated by those in the art, the subject may be an animal.

The terms “therapeutically effective amount”, “effective dose”, “therapeutically effective dose”, “effective amount,” or the like refer to the amount of a subject compound that will elicit the biological or medical response in a tissue, system, animal or human that is being sought by administering said compound. Generally, the response is either amelioration of symptoms in a patient or a desired biological outcome. In some embodiments, such amount should be sufficient to inhibit a c-kit kinase.

In some embodiments, an effective amount of a c-kit inhibiting compound of the invention is an amount that ranges from about 10 pg to 500 mg, e.g., from about 10 pg to 50 pg, from about 50 pg to 150 pg, from about 150 pg to 250 pg, from about 250 pg to 500 pg, from about 500 pg to 750 pg, from about 750 pg to 1 ng, from about 1 ng to 10 ng, from about 10 ng to 50 ng, from about 50 ng to 150 ng, from about 150 ng to 250 ng, from about 250 ng to 500 ng, from about 500 ng to 750 ng, from about 750 ng to 1 mg, from about 1 pg to 10 pg, from about 10 pg to 50 pg, from about 50 pg to 150 pg, from about 150 pg to 250 pg, from about 250 pg to 500 pg, from about 500 pg to 750 pg, from about 750 pg to 1 mg, from about 1 mg to 50 mg, from about 1 mg to 100 mg, from about 50 mg to 100 mg, from about 100 mg to 200 mg, from about 200 mg to 300 mg, from about 300 mg to 400 mg, from about 400 mg to 500 mg, or from about 100 mg to 500 mg. The amount can be a single dose amount or can be a total daily amount. The total daily amount can range from about 10 pg to 100 mg, or can range from about 100 mg to 500 mg, or can range from about 500 mg to 1000 mg. In some embodiments, an effective amount of a c-kit inhibiting compound of the invention is about 300 mg. In some embodiments, an effective amount of a c-kit inhibiting compound of the invention is about 500 mg. In some embodiments, an effective amount of a c-kit inhibiting compound of the invention is about 1 g.

### Definitions

As used herein, the term “about”, when used in reference to an amount refers to the stated value ±10% of said value. In some embodiments, “about” refers to the stated value ±5% of said value, ±2% of said value, or ±1% of said value.

As used herein, the terms “administer,” “administering,” and “administration,” refer to any method which, in sound medical practice, delivers a provided composition, or an active agent contained therein, to a subject in such a manner as to provide a therapeutic effect.

As used herein, the phrases an “effective amount” or a “therapeutically effective amount” of an active agent or ingredient, or pharmaceutically active agent or ingredient, refer to an amount of the pharmaceutically active agent sufficient enough to have a therapeutic effect upon administration. Effective amounts of the pharmaceutically active agent will vary with the kind of pharmaceutically active agent chosen, the particular condition or conditions being treated, the severity of the condition, the duration of the treatment, the specific components of the composition being used, and like factors. Generally, the response is either amelioration of symptoms in a patient or a desired biological outcome. In some embodiments, such amount should be sufficient to inhibit a c-kit kinase and treat a c-kit kinase related disease or disorder.

As used herein, the phrase “pharmaceutically acceptable salts” refers to salts of certain ingredient(s) which possess the same activity as the unmodified compound(s) and which are neither biologically nor otherwise undesirable. A salt can be formed with, for example, organic or inorganic acids. Such suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, and naturally and synthetically derived amino acids.

As used herein the term “preservative” refers to any known pharmaceutically acceptable preservative that functions by inhibiting bacteria, fungi, yeast, mold, other microbe, and/or by inhibiting oxidation. Suitable preservatives include but are not limited to antimicrobial agents and/or antioxidants. Suitable antimicrobial agents can include but are not limited to benzoates, benzyl alcohol, sodium benzoate, sorbates, propionates, and nitrites. Suitable antioxidants can include but are not limited to vitamin C, butylated hydroxytoluene (BHT), sulphites, and vitamin E. Other such preservatives for use in the present invention are described above and herein.

The term “prevent,” “preventing,” or “prevention,” as used herein refers to any reduction, no matter how slight, of a subject's predisposition or risk for developing a condition, disease, disorder or symptom thereof. For purposes of prevention, the subject is any subject, and preferably is a subject that is at risk for, or is predisposed to, developing a condition, disease, disorder. The term “prevention” includes either preventing the onset of a clinically evident condition, disease, disorder altogether or preventing the onset of a pre-clinically evident condition, disease, disorder in individuals at risk. This includes prophylactic treatment of subjects at risk of developing condition, disease, disorder.

As used herein, the term “solvent” refers to any pharmaceutically acceptable medium which is a liquid at ambient temperature, in which one or more solutes can be dissolved, or one or more substances can be partially dissolved or suspended. Numerous solvents are well known in the chemical and pharmaceutical arts and are contemplated herein and below.

The phrase “substantially pure” as used herein refers to an individual compound form, which is substantially devoid of all other forms, as well as degradation products of a form, and any residual solvent, and is at least 85% pure on a % weight basis, unless otherwise specified. The compound form can have at least 90% purity on a % weight basis, at least 93% purity on a % weight basis, at least 95% purity on a % weight basis, or at least 97%, 98%, 99%, or 99.5% purity on a % weight basis.

As used herein, “subject” or “individual” or “animal” or “patient” or “mammal,” refers to any subject, particularly a mammalian subject, for whom diagnosis, prognosis, or therapy is desired, for example, a human.

As used herein, a “treatment” or “treating” of a disease, disorder, or condition encompasses alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of symptoms associated therewith, provide improvement to a patient or subject's quality of life, or delay or inhibit the onset of a disease, disorder, or condition.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As used herein, all percentages are by weight of the total composition (i.e., wt %), unless otherwise specified.

Any concentration ranges, percentage range, or ratio range recited herein are to be understood as expressly disclosing and including any concentrations, percentages or ratios of any integer within that range and fractions thereof, such as one tenth and one hundredth of an integer, and any sub-range falling within a range, unless otherwise indicated.

Any number range recited herein relating to any physical feature, including for example, polymer subunits, size or thickness, are to be understood as expressly disclosing and including any integer or fraction of an integer within a disclosed range, or any sub-range within a disclosed range, unless otherwise indicated.

For the purpose of clarity, any element or feature of any method or composition or process described herein, can be combined with any other element or feature of any other method or composition or process described herein.

Other terms as used herein are meant to be defined by their well-known meanings in the art.

All features of each of the aspects of the disclosure apply to all other aspects mutatis mutandis. Each of the references referred to herein, including but not limited to patents, patent applications and journal articles, is incorporated by reference herein as though fully set forth in its entirety,

In order that the disclosure described herein may be more fully understood, the following examples are set forth. It should be understood that these examples are for illustrative purposes only and are not to be construed as limiting this disclosure in any manner.

### EXAMPLES

As depicted in the Examples below, in certain exemplary embodiments, compounds and compositions are prepared according to the following general procedures. The following examples are illustrative of the present pharmaceutical compositions and are not intended to be limitations thereon.

### Materials and Methods

Preparation of Free Base Forms a, H^{A}, H^{B}, and D and HCl Salt Form I of Compound 1

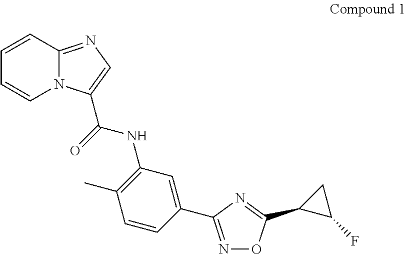 [SMILES: Cc1ccc(-c2noc([C@H]3C[C@@H]3F)n2)cc1NC(=O)c1cnc2ccccn12]

### Form a of Compound 1

Form A of compound 1 was prepared as disclosed in PCT/CN2020/090060, which is incorporated by reference herein:

Procedure A: About 2.0 g of amorphous compound 1 (as prepared in Example F110 of WO 2013/033070 A1) was dissolved in 40 mL of IPA at 70° C. and mechanically stirred for 3 hours, resulting in a clear solution. The solution was then cooled to rt and continually stirred overnight. Precipitate formed overnight and was filtered and washed with IPA and dried overnight at 60° C. under vacuum. Characterization of the resulting material demonstrated crystalline Form A of Compound 1 free base.

### Form H^{A} of Compound 1

Form H^{A} of compound 1 was prepared as disclosed in PCT/CN2020/090060, which is incorporated by reference herein:

Procedure A: About 200 mg of Form A of compound 1 was dissolved in 3.0 mL of MeOH/H₂O (1:1, v/v) and stirred at 1000 rpm at RT for 5 days. The suspension was centrifuged and the solids were dried under vacuum. Characterization of the resulting material demonstrated crystalline Form H^{A} of Compound 1 free base.

### Form H^{B} of Compound 1

Form H^{B} of compound 1 was prepared as disclosed in PCT/CN2020/090060, which is incorporated by reference herein:

Procedure A: About 10 mg of Form A of compound 1 was placed in a vial containing water for 2 weeks. The solid was isolated from the suspension and it was observed that Form A had been converted to Form H^{B}. Characterization of the resulting material demonstrated crystalline Form H^{B} of Compound 1 free base.

### Form D of Compound 1

Form D of compound 1 was prepared as follows:

Procedure A: 50° C. Slurry Screen—About 20 mg of Form A of compound 1 was suspended in 0.5 mL of IPA in an HPLC vial. The sample was stirred magnetically (˜1000 rpm) for about 7 days at 50° C., the remaining solids were isolated for XRPD analysis.

Procedure B: 50° C. Slurry Screen—About 20 mg of Form A of compound 1 was suspended in 0.5 mL of CPME in an HPLC vial. The sample was stirred magnetically (˜1000 rpm) for about 7 days at 50° C., the remaining solids were isolated for XRPD analysis.

Procedure C: Anti-solvent addition screen—About 20 mg of Form A of compound 1 was dissolved in DCM to obtain a clear solution and the solution was magnetically stirred (˜1000 rpm) followed by addition of MTBE until precipitate appeared. The obtained precipitate was isolated for XRPD analysis.

Procedure D: Anti-solvent addition screen—About 20 mg of Form A of compound 1 was dissolved in pyridine to obtain a clear solution and the solution was magnetically stirred (˜1000 rpm) followed by addition of EtOAc until precipitate appeared. The obtained precipitate was isolated for XRPD analysis.

Procedure E: 204.5 mg of Form A of compound 1 was suspended in 3.0 mL IPA and stirred at 1000 rpm at RT for 5 days. The suspension was centrifuged and the solids were dried under vacuum. Characterization of the resulting material demonstrated crystalline Form D of Compound 1 free base.

### Form I of Compound 1

Procedure A: 2008.0 mg of compound 1 free base was added to a 100-mL bottle, followed by addition of 20 mL of isopropanol to obtain a suspension. 549.3 mg of HCl (36 wt % solution) was added to a 20 mL vial, followed by addition of 20 mL of isopropanol to obtain an HCl solution. The HCl solution was added to the 100-mL bottle and the mixture was stirred (500 rpm) at room temperature for 3 days. The mixture was filtered and the collected solids were dried under vacuum at room temperature overnight. The obtained precipitate was submitted for XRPD analysis.

### Characterization Methods

### PLM

Polarized light microscopy (PLM) was conducted using a Nikon LV100POL equipped with a 5 megapixel CCD and either a 20× and 50× physical lens.

### XRPD

X-ray powder diffraction (XRPD) analysis was conducted using a Bruker D8 Advance diffractometer with the following parameters:
Tube: Cu: K-Alpha (λ=1. 54060\acute{Å}).
Generator: Voltage: 40 kV; Current: 40 mA;
Scan Scope: 3 to 40 deg, or 2 to 40 deg;
Scanning rate: 10 deg./min, or 19 deg./min;
Sample rotation speed: 15 rpm

### PSD for Nanosuspension Samples

Particle size distribution (PSD) analysis was conducted on nanosuspension samples using a Zeta Potential & Particle Sizer (ZPPS) (Nicomp 380/ZLS, Nicomp) using the following parameters:
Run time: 2-4 mins
Average Intensity: 250-350 kHz
Wavelength=632.8 nm
Temperature: 23° C.
Viscosity=0.933 cp
Index of Ref.=1.333
Calculation mode: Intensity-weighted Gaussian Distribution Analysis

### UPLC

Ultra-performance liquid chromatography (UPLC) was used to measure the concentration of Compound 1 in each nanosuspension and the purity of the Compound 1. The following parameters were used:

| | Instrument | | |
|---|---|---|---|
| | Agilent 1290 infinityII | | |
| | Column | | |
| | ACQUITY UPLC BEH C18, | | |
| | 1.7 um 2.1*50 mm (PDS-HPLC-286) | | |
| | Time | A%: 10 mM | B%: |
| | (min) | aq. NH₄OAC | ACN |
| Gradient | 0 | 90 | 10 |
| | 4.0 | 45 | 55 |
| | 5.2 | 0 | 100 |
| | 5.21 | 90 | 10 |
| | 6.0 | 90 | 10 |

| | |
|---|---|
| Injection volume | 1 μL |
| Flow rate | 0.5 mL/min |
| Column Temp. | 30° C. |
| Wavelength | 240 nm |
| Diluent | ACN:H₂O = 7:3 |

### HPLC

High performance liquid chromatography (HIPLC) was used to measure the purity of the Compound 1 in certain samples. The following parameters were used:

| | Instrument | | |
|---|---|---|---|
| | Agilent 1260 infinityII | | |
| | Column | | |
| | XBridge C18, 3.5 um 3.0*150 mm | | |
| | (PDS-HPLC-367) | | |
| | Time | A%: 10 mM | B%: |
| | (min) | aq. NH₄OAc | ACN |
| Gradient | 0 | 80 | 20 |
| | 18 | 5 | 95 |
| | 21 | 5 | 95 |
| | 21.1 | 80 | 20 |
| | 27 | 80 | 20 |

| | |
|---|---|
| Injection volume | 2 μL |
| Flow rate | 0.8 mL/min |
| Column Temp. | 30° C. |
| Wavelength | 248 nm |
| Diluent | ACN:H₂O = 1:1 |

### Materials

Sodium dodecyl sulfate (SDS) 99.000 was purchased from SIGMA. PVP-VA64 was purchased from BASF.

### Example 1: Nanosuspension Preparation by Planetary Ball Mill

### Sample Preparation

Four nanosuspension formulations of Compound 1 with different vehicle formulations as shown in Table 2 were prepared at a target Compound 1 concentration of 100 mg/mL using the following procedure. The appropriate vehicle compositions were added into 250-mL glass vials and dissolved completely under magnetic stirring at 700 rpm and sonicated to obtain a clear vehicle solution. The vehicle solutions were mixed with 0.2 mm zirconia beads at a ratio of 3:1 (v/v) in a 125-mL grinding chamber. Compound 1 was added into each vial in the appropriate amount. The suspensions were ground at 300 rpm by planetary ball mill (PM400). Particle size distribution (PSD) was monitored during the milling process. When D₅₀ reached about 200 nm or D₅₀ had no obvious change over time, the milling process was halted. The suspensions were diluted with vehicle, as needed, to obtain homogenous nanosuspensions with the target concentration of 100 mg/mL of Compound 1.

TABLE 2
Formulations and parameters of nanosuspensions preparation by PM400

| Formulation | 1A | 1B | 1C | 1D |
|---|---|---|---|---|
| Form | Free base | Free base | HCl salt | Free base |
| | hydrate | hydrate | | anhydrate |
| | Form H^{A} | Form H^{A} | | Form A |
| Vehicle | PVP-VA64/ | PVPK30/ | PVP-VA64/ | PVP-VA64/ |
| (by weight) | SDS/water = | SDS/water = | SDS/water = | SDS/water = |
| | 3/0.1/86.9 | 2/0.1/87.9 | 3/0.1/86.9 | 3/0.1/86.9 |
| Milling ratios | API/PVP- | API/PVPK30/ | API/PVP- | API/PVP- |
| (by weight) | VA64/SDS/ | SDS/ | VA64/SDS/ | VA64/SDS/ |
| | water = | water = | water = | water = |
| | 10/3/0.1/86.9 | 10/2/0.1/87.9 | 10/3/0.1/86.9 | 10/3/0.1/86.9 |
| Volume of | 45 | 37.5 | 27.75 | 37.5 |
| vehicle | | | | |
| Milling time | 2.5 | 7.3 | 11.2 | 10.9 |
| (h) | | | | |
| Final D₅₀ | 202.8 | 217.2 | 1044.5 | 292.0 |
| (nm) | | | | |

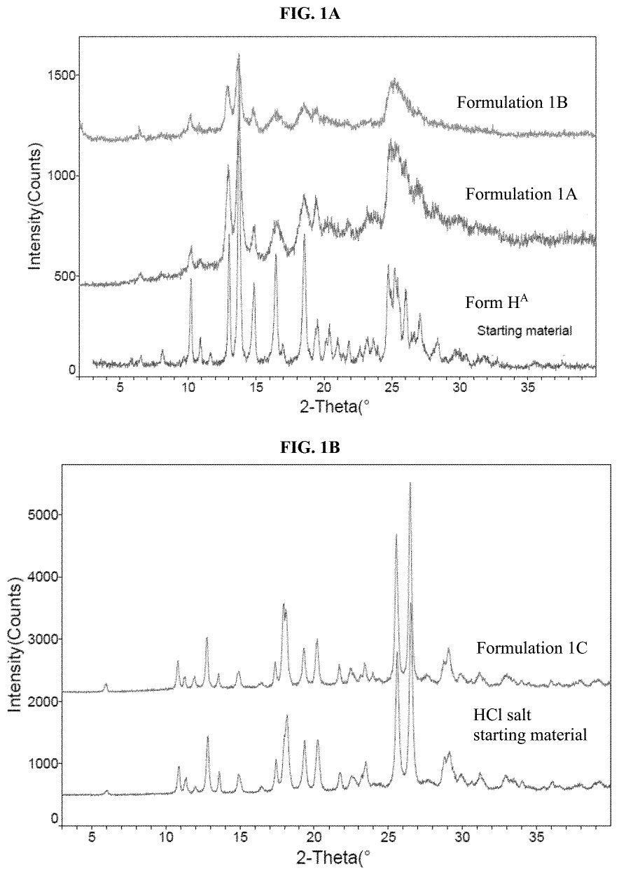

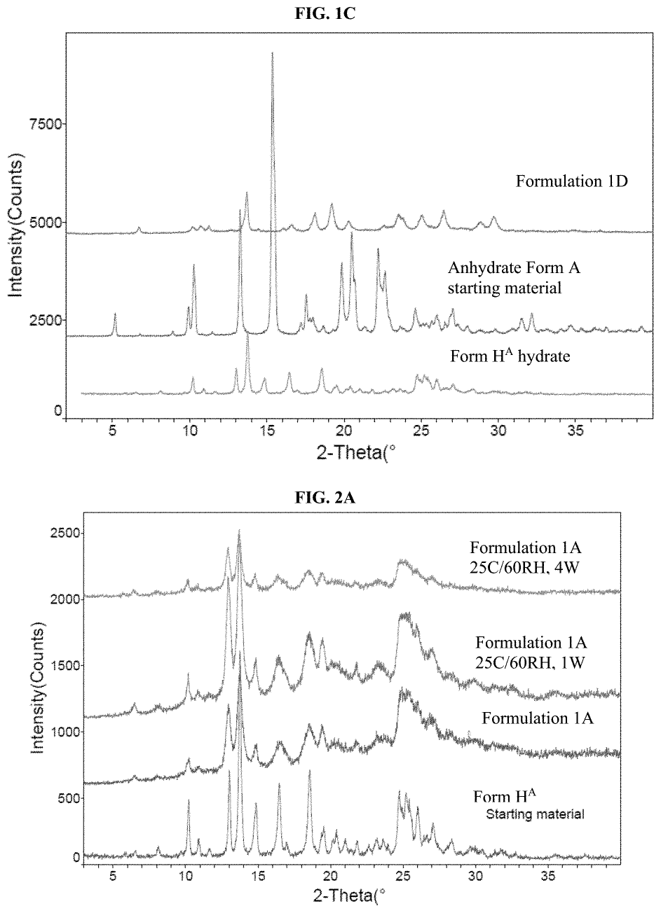

Nanosuspension formulations were characterized based on pH, PLM/SEM, XRPD, PSD and purity (HPLC). The results are reported below in Tables 3A and 3B. XRPD spectra comparing the starting material and the nanosuspensions are shown in FIGS. 1A-1C.

TABLE 3A
Characterization of nanosuspensions prepared by PM400

| | | PSD | | | | | |
|---|---|---|---|---|---|---|---|
| | | Initial | | | | After 1 day or 3 days | |
| Formula | | D₅₀ | | Variance | | D₅₀ | Variance |
| No. | Appearance | (nm) | | (P.I.) | | (nm) | (P.I.) |
| 1A | Homogenous tan | 211.4 | | 0.198 | | 223.4 | 0.315 |
| | suspension | | | | | (1 day) | (1 day) |
| 1B | Homogenous tan | 1 | 219.2 | 1 | 0.294 | 211.1 | 0.236 |
| | suspension | 2 | 205.8 | 2 | 0.289 | (3 days) | (3 days) |
| | | 3 | 214.3 | 3 | 0.299 | | |
| | | Avg. | 213.1 | N/A | N/A | | |
| 1C | Homogenous off- | 1 | 1580.4 | 1 | 0.701 | N/A | N/A |
| | white suspension | 2 | 1476.6 | 2 | 0.579 | | |
| | | Avg. | 1528.5 | N/A | N/A | | |

| | | | | | | | |
|---|---|---|---|---|---|---|---|
| 1D | Homogenous white | 301.4 | 0.288 | 1 | 645.5 | 1 | 0.184 |
| | suspension | | | 2 | 696.6 | 2 | 0.130 |
| | | | | Avg. | 670.1 | N/A | N/A |

TABLE 3B
Characterization of nanosuspensions prepared by PM400

| | | | | HPLC test | | | |
|---|---|---|---|---|---|---|---|
| | | | | Conc. | | Purity | |
| Formula No. | XRPD | PLM | pH | (mg/mL) | | (%) | |
| 1A | No form change | Birefringence | 7.12 | 97.1 | | 97.61 | |
| 1B | No form change | Birefringence | 6.90 | 1 | 100.9 | 1 | 97.62 |
| | | | | 2 | 97.3 | 2 | 97.59 |
| | | | | Avg. | 99.1 | Avg. | 97.61 |
| 1C | No form change | Birefringence | 1.59 | 93.3 | | 97.65 | |
| 1D | Mixture of forms | Birefringence | 5.99 | 1 | 98.3 | 1 | 98.53 |
| | | | | 2 | 96.8 | 2 | 98.54 |
| | | | | Avg. | 97.6 | Avg. | 98.54 |

### Form H^{A} Hydrate Nanosuspension Formulations 1A and 1B

According to PLM images and XRPD patterns (FIG. 1A), Formulations 1A 1B were crystalline with the same pattern as Form H^{A} starting material, but the crystallinity decreased dramatically after ball milling. The particle size distribution results showed D₅₀ of about 210 nm with a polydispersity index (PI) less than 0.3. The particle D₅₀ showed no obvious changes after 1 day or 3 days. According to the HPLC results, the concentration of Compound 1 was 97.1 mg/mL and 99.1 mg/mL for Formulations 1A and 1B respectively, quite close to the target concentration of 100 mg/mL. The Compound 1 purity of the two nanosuspensions were determined to be 97.61%, which were nearly identical to the Compound 1 starting material (starting material purity was 97.62%).

### HCl Salt Nanosuspension Formulation 1C

According to PLM images and XRPD pattern (FIG. 1), the Formulation 1C nanosuspension was crystalline with the same pattern as the HCl salt starting material. The particle size distribution results showed a D₅₀ of about 1500 nm with a PI variance greater than 0.3. The Compound 1 concentration was 93.3 mg/mL based on HPLC testing. The Compound 1 purity was determined to be 97.65%, which was nearly identical to the starting material (starting material purity was 97.5% Error! Reference source not found).

### Form A Anhydrate Nanosuspension Formulation 1D

Formulation 1D showed higher crystallinity than Formulations 1A or 1B according to PLM images and XRPD pattern (FIG. 1C). However, the pattern indicated the suspension contained a mixture of hydrate and anhydrate forms based on characteristic peaks not present in the original material. The particle size distribution results showed a D₅₀ of 301.4 nm with a PI variance less than 0.3, but D₅₀ increased to 670 nm after 1 day. The HPLC test results displayed that the concentration was 97.6 mg/mL and the purity was 98.54%, which was nearly identical to the starting material (starting material purity was 98.36%).

### 1-Week and 4-Week Stability Studies

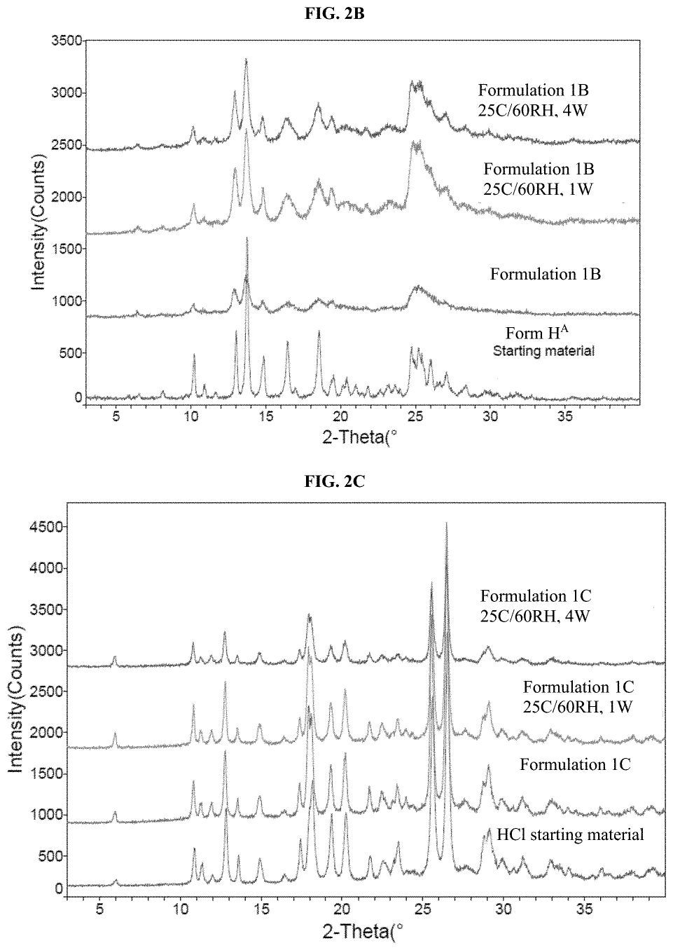

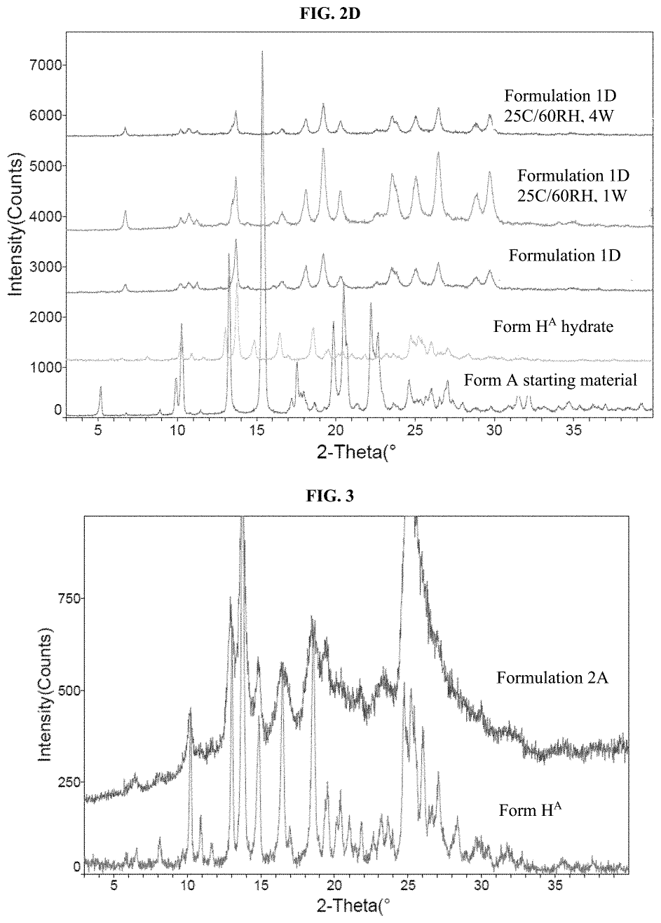

The chemical and physical stability of the nanosuspension formulations were studied under 25° C./60% RH (sealed and protected from light) conditions. The nanosuspensions were transferred into 4-mL glass vials with locking lids. The vials were covered by aluminum foil and stored in a stability chamber at 25° C./60% RH for 1 week or 4 weeks. The 1-week and 4-week stability study results for Formulations 1A-1D are reported below in Table 4. XRPD spectra comparing the starting material and the nanosuspensions after the 1 week and 4 week studies are shown in FIGS. 2A-2D.

### Form H^{A} Hydrate Nanosuspension Formulations 1A and 1B

PLM and XRPD patterns (FIGS. 2A and 2B) for Formulations 1A and 1B stored under 25° C./60% RH (sealed) for 1 week showed that the Compound 1 material remained crystalline with the same pattern as starting material. The particle size distribution results showed no change in D₅₀ in Formulation 1A after 1 week, but the D₅₀ of the particles in Formulation 1B increased to 532.2 nm. The purity HPLC results showed that both Formulations 1A and 1B were chemically stable.

PLM and XRPD patterns (FIGS. 2A and 2B) for Formulations 1A and 1B stored under 25° C./60% RH (sealed) for 4 weeks showed that the Compound 1 material remained crystalline with the same pattern as starting material. The particle size distribution results showed no change in D₅₀ in both Formulations 1A and 1B after 4 weeks. The purity HPLC results showed that both Formulations 1A and 1B were chemically stable.

### HCl Salt Nanosuspension Formulation 1C

PLM and XRPD patterns (FIG. 2C) for Formulation 1C stored under 25° C./60% RH (sealed) for 1 week showed that the Compound 1 material remained crystalline with same pattern as starting material. The particle size D₅₀ increased to ˜3500 nm with variance greater than 0.3, indicating an agglomeration of particles. The purity HPLC results showed that the Compound 1 in Formulation 1C was stable and the purity remained almost the same as starting material.

PLM and XRPD patterns (FIG. 2C) for Formulation 1C stored under 25° C./60% RH (sealed) for 4 weeks showed that the Compound 1 material remained crystalline with same pattern as starting material. The particle size D₅₀ increased to ˜2500 nm with variance greater than 0.3, indicating an agglomeration of particles. The purity HPLC results showed that the Compound 1 in Formulation 1C was stable and the purity remained almost the same as starting material.

### Form A Anhydrate Nanosuspension Formulation 1D

PLM and XRPD patterns (FIG. 2D) for Formulation 1D stored under 25° C./60% RH (sealed) for 1 week showed that the Compound 1 material remained crystalline but appeared to be a mixture of hydrate and anhydrate forms based on characteristic peaks. The particle size distributions results showed no appreciable change in D₅₀. The purity HPLC results showed that the Compound 1 in Formulation 1D was stable and the purity remained almost the same as starting material.

PLM and XRPD patterns (FIG. 2D) for Formulation D stored under 25° C./60% RH (sealed) for 4 weeks showed that the Compound 1 material remained crystalline but appeared to be a mixture of hydrate and anhydrate forms based on characteristic peaks. The particle size distributions results showed no appreciable change in D₅₀. The purity HPLC results showed that the Compound 1 in Formulation 1D was stable and the purity remained almost the same as starting material.

TABLE 4A
1 week and 4 week stability data

| | | | PSD | | | |
|---|---|---|---|---|---|---|
| Formula | | | D₅₀ | | Variance | |
| No. | Conditions | Appearance | (nm) | | (P.I.) | |
| 1A | Initial | Homogenous tan | 211.4 | | 0.198 | |
| | | suspension | | | | |
| | 25° C./60% RH, | Homogenous tan | 226.2 | | 0.264 | |
| | sealed, 1 W | suspension | | | | |
| | 25° C./60% RH, | Homogenous tan | 234.0 | | 0.335 | |
| | sealed, 4 W | suspension | | | | |
| 1B | Initial | Homogenous tan | 1 | 219.2 | 1 | 0.294 |
| | | suspension | 2 | 205.8 | 2 | 0.289 |
| | | | 3 | 214.3 | 3 | 0.299 |
| | | | Avg. | 213.1 | N/A | N/A |
| | 25° C./60% RH, | Homogenous tan | 532.2 | | 0.276 | |
| | sealed, 1 W | suspension | | | | |
| | 25° C./60% RH, | Homogenous tan | 215.0 | | 0.286 | |
| | sealed, 4 W | suspension | | | | |
| 1C | Initial | Homogenous off-white | 1 | 1580.4 | 1 | 0.701 |
| | | suspension | 2 | 1476.6 | 2 | 0.579 |
| | | | Avg. | 1528.5 | N/A | N/A |

| | | | | |
|---|---|---|---|---|
| | 25° C./60% RH, | Homogenous off-white | 3515.1 | 1.040 |
| | sealed, 1 W | suspension | | |
| | 25° C./60% RH, | Homogenous off-white | 2444.9 | 0.487 |
| | sealed, 4 W | suspension | | |
| 1D | Initial | Homogenous white | 301.4 | 0.288 |
| | | suspension | | |
| | 25° C./60% RH, | Homogenous white | 269.8 | 0.272 |
| | sealed, 1 W | suspension | | |
| | 25° C./60% RH, | Homogenous white | 315.2 | 0.365 |
| | sealed, 4 W | suspension | | |

TABLE 4B
1 week and 4 week stability data

| | | | | | HPLC | | | |
|---|---|---|---|---|---|---|---|---|
| Formula | | | | | Conc. | | Purity | |
| No. | Conditions | XRPD | PLM | pH | (mg/mL) | | (%) | |
| 1A | Initial | No form | Birefringence | 7.12 | 97.1 | | 97.61 | |
| | | change | | | | | | |
| | 25° C./60% RH, | No form | Birefringence | 6.41 | N/A | | 97.66 | |
| | sealed, 1 W | change | | | | | | |
| | 25° C./60% RH, | No form | Birefringence | 6.88 | N/A | | 97.72 | |
| | sealed, 4 W | change | | | | | | |
| 1B | Initial | No form | Birefringence | 6.90 | 1 | 100.9 | 1 | 97.62 |
| | | change | | | 2 | 97.3 | 2 | 97.59 |
| | | | | | Avg. | 99.1 | Avg. | 97.61 |
| | 25° C./60% RH, | No form | Birefringence | 7.39 | N/A | | 97.58 | |
| | sealed, 1 W | change | | | | | | |
| | 25° C./60% RH, | No form | Birefringence | 7.04 | N/A | | 97.72 | |
| | sealed, 4 W | change | | | | | | |
| 1C | Initial | No form | Birefringence | 1.59 | 93.3 | | 97.65 | |
| | | change | | | | | | |
| | 25° C./60% RH, | No form | Birefringence | 1.56 | N/A | | 97.61 | |
| | sealed, 1 W | change | | | | | | |
| | 25° C./60% RH, | No form | Birefringence | 1.60 | N/A | | 97.32 | |
| | sealed, 4 W | change | | | | | | |
| 1D | Initial | Mixture | Birefringence | 5.99 | 1 | 98.3 | 1 | 98.53 |
| | | | | | 2 | 96.8 | 2 | 98.54 |
| | | | | | Avg. | 97.6 | Avg. | 98.54 |

| 25° C./60% RH, | Mixture | Birefringence | 6.50 | N/A | 98.64 |
|---|---|---|---|---|---|
| sealed, 1 W | | | | | |
| 25° C./60% RH, | Mixture | Birefringence | 5.84 | N/A | 98.48 |
| sealed, 4 W | | | | | |

### Example 2: Nanosuspension Preparation Using Dynomill

### Sample Preparation

Two nanosuspension formulations of Compound 1, one with Form H^{A} and the other with Form A, were prepared using Dynomill at target Compound 1 concentrations of 100 mg/mL using the following procedure. Vehicle formulations containing PVP-VA64/SDS/water=3/0.1/86.9 was added into 250-mL glass vials and dissolved completely by magnetically stirring at 700 rpm followed by sonication to obtain clear vehicle solutions. The vehicle solutions were mixed with 50 mL 0.3 mm zirconia beads in 80-mL grinding chambers. Appropriate amounts of Compound 1 were added to the chambers. The suspensions were then ground by dynomill. Particle size distribution (PSD) was monitored during the milling process. When D₅₀ of the Compound 1 particles reached about 200 nm or the D₅₀ had no obvious change over time, the milling process was halted. The suspensions were diluted with vehicle to obtain homogenous nanosuspensions with the target Compound 1 concentration of 100 mg/mL.

TABLE 5
Composition and parameters of
nanosuspension preparation by dynomill

| Formulation | 2A | 2B |
|---|---|---|
| Form | Free base | Free base |
| | hydrate | anhydrate |
| | Form H^{A} | Form A |
| Vehicle | PVP-VA64/ | PVP-VA64/ |
| (by weight) | SDS/water = | SDS/water = |
| | 3/0.1/86.9 | 3/0.1/86.9 |
| Milling Ratios | API/PVP-VA64/ | API/PVP-VA64/ |
| (by weight) | SDS/water = | SDS/water = |
| | 10/3/0.1/86.9 | 10/3/0.1/86.9 |
| API quantity | 6.5 | 12 |
| (g) | | |
| Vehicle | 50 | 90 |
| volume (mL) | | |
| Milling time | 3 | 6 |
| (h) | | |

### Form H^{A} Hydrate Nanosuspension Formulation 2A

According to PLM images and XRPD patterns (FIG. 3), Formulation 2A contained crystalline Compound 1 with the same pattern as Form H^{A} starting material, but the crystallinity decreased dramatically after dynomilling. The particle size distribution results showed a D₅₀ of ˜160 nm, with a variance lower than 0.3. After 1 day, there was no observable change in particle D₅₀ in Formulation 2A. The concentration of Compound 1 in Formulation 2A was determined to be 94.6 mg/mL by HPLC and the purity was determined to be 97.50%, nearly the same as the starting material (starting material purity was 97.62%).

### Form A Anhydrate Nanosuspension Formulation 2B

The particle size distribution results showed a D₅₀ of ˜220 nm, with a variance lower than 0.3. The concentration of Compound 1 in Formulation 2B was determined to be 101.8 mg/mL by HPLC and the purity was determined to be 98.5300, nearly the same as the starting material (starting material purity was 98.360%).

TABLE 6A
Characterization of nanosuspensions prepared by dynomill

| | | PSD | | | |
|---|---|---|---|---|---|
| Formulation | Appearance | D₅₀ (nm) | P.I. | XRPD | PLM |
| 2A | Homogenous | 164.4 | 0.259 | No form | Bire- |
| | tan | | | change | fringence |
| | suspension | | | | |
| 2B | Homogenous | 223.8 | 0.107 | N/A | N/A |
| | white | | | | |
| | suspension | | | | |

TABLE 6B
Characterization of
nanosuspensions prepared by dynomill

| | | HPLC test | | | |
|---|---|---|---|---|---|
| | | Assay | | Purity | |
| Formulation | pH | (mg/mL) | | (%) | |
| 2A | 6.73 | 1 | 94.7 | 1 | 97.51 |
| | | 2 | 94.6 | 2 | 97.49 |
| | | Avg. | 94.6 | Avg. | 97.50 |
| 2B | N/A | 1 | 100.4 | 1 | 98.53 |
| | | 2 | 103.3 | 2 | 98.53 |
| | | Avg. | 101.8 | Avg. | 98.53 |

### 1-Week and 4-Week Stability Studies on Formulation 2A

The chemical and physical stability of the dynomill prepared Formulation 2A nanosuspension was studied under 4° C. (sealed), 25° C./60% RH (sealed and protected from light) conditions. The nanosuspensions were transferred into 4-mL glass vials with locking lids. The vials were covered by aluminum foil and stored in a stability chamber at 4° C. or 25° C./60% RH for 1 week or 4 weeks. The 1-week and 4-week stability study results for Formulation 2A is reported below in Table 7.

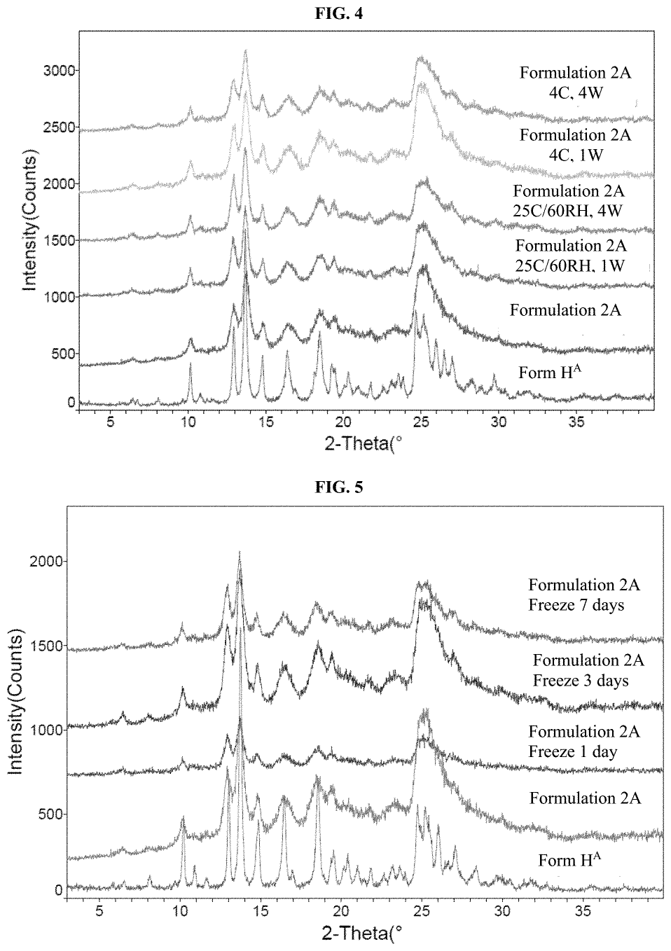

PLM and XRPD patterns (FIG. 4) for Formulation 2A under both conditions after both 1 week and 4 weeks showed that the Compound 1 material remained crystalline with the same pattern as starting material. The particle size distribution results showed that there was no observable change in particle D₅₀ after either 1 week or 4 weeks. The purity HPLC results showed that the Compound 1 in Formulation 2A was stable and the purity remained almost the same as starting material over both the 1 week study and the 4 week study. The appearance of all materials remained a homogeneous tan suspension.

TABLE 7
1 week and 4 week stability data for Formulation 2A

| | PSD | | | | | HPLC | | | |
|---|---|---|---|---|---|---|---|---|---|
| | D₅₀ | | | | | Conc. | | Purity | |
| Conditions | (nm) | P.I. | XRPD | PLM | pH | (mg/mL) | | (%) | |
| Initial | 164.4 | 0.259 | No form | Birefringence | 6.73 | 1 | 94.7 | 1 | 97.51 |
| | | | change | | | 2 | 94.6 | 2 | 97.49 |
| | | | | | | Avg | 94.6 | Avg | 97.50 |

| | | | | | | | |
|---|---|---|---|---|---|---|---|
| 4° C., sealed, 1 W | 175.0 | 0.284 | No form | Birefringence | 6.98 | N/A | 97.66 |
| | | | change | | | | |
| 4° C., sealed, 4 W | 189.5 | 0.309 | No form | Birefringence | 6.94 | N/A | 97.51 |
| | | | change | | | | |
| 25° C./60% RH, | 171.2 | 0.270 | No form | Birefringence | 7.00 | N/A | 97.67 |
| sealed, 1 W | | | change | | | | |
| 25° C./60% RH, | 183.3 | 0.254 | No form | Birefringence | 7.29 | N/A | 97.45 |
| sealed, 4 W | | | change | | | | |

### Freeze/Thaw Assessment

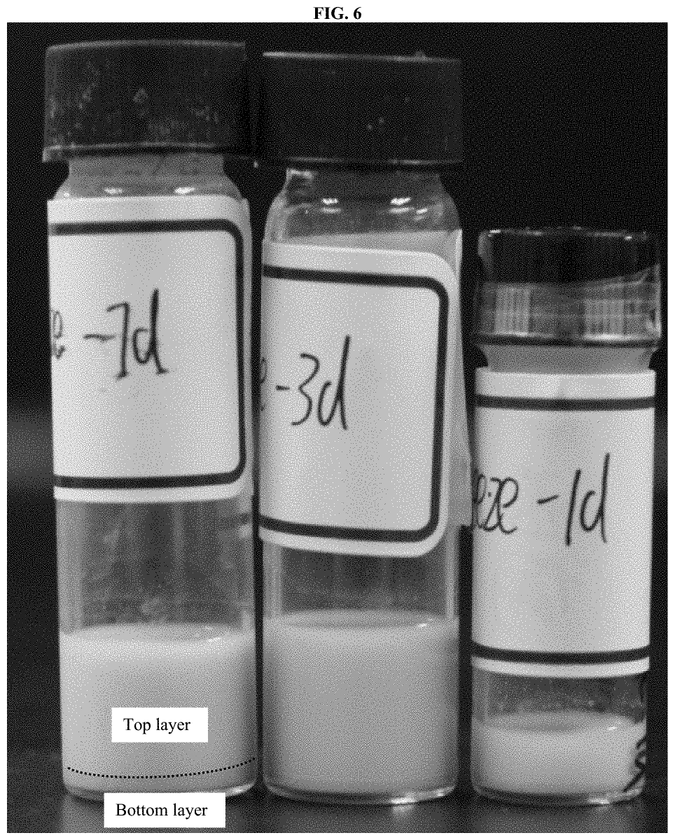

Samples of nanosuspension formulation 2A were frozen under dry ice and stored in a frozen state for 1 day, 3 days or 7 days and then allowed to thaw at room temperature (˜25° C.) under sonication. The frozen samples were thawed over several hours. When no ice was observed in the suspension, and the suspension demonstrated good flowability, the suspensions were sonicated for an additional 15-30 mins until a fully homogenous suspension was observed by visual inspection. Sedimentation occurred for each sample after left to stand for 0.5 hours, showing two distinct layers in the suspension after a total of 1 hour (FIG. 6).

The thawed formulation 2A suspensions were investigated by pH measurement, PLM, XRPD, and HPLC to determine purity. The characterization results are shown below in Table 8. The D₅₀ particle size of all samples after freezing for 1 day, 3 days and 7 days had a small increase to about 200 nm with a variance greater than 0.3. The PLM and XRPD (FIG. 5) results indicated all samples were crystalline with the same pattern as the starting material. The HPLC results indicated that the purity of the Compound 1 in the thawed samples was almost the same as the initial suspension and starting material.

TABLE 8
Characterization of freeze/thawed samples of Formulation 2A

| | PSD | | | | | HPLC | | | |
|---|---|---|---|---|---|---|---|---|---|
| | D₅₀ | Variance | | | | Conc. | | Purity | |
| Conditions | (nm) | (P.I.) | XRPD | PLM | pH | (mg/mL) | | (%) | |
| Initial | 164.4 | 0.259 | No | Birefringence | 6.73 | 1 | 94.7 | 1 | 97.51 |
| | | | form | | | 2 | 94.6 | 2 | 97.49 |
| | | | change | | | Avg. | 94.6 | Avg. | 97.50 |

| | | | | | | | |
|---|---|---|---|---|---|---|---|
| Freeze for | 199.0 | 0.594 | No | Birefringence | 7.17 | N/A | 97.76 |
| 1 day | | | form | | | | |
| | | | change | | | | |
| Freeze for | 172.8 | 0.475 | No | Birefringence | 6.81 | N/A | 97.74 |
| 3 day | | | form | | | | |
| | | | change | | | | |
| Freeze for | 231.1 | 0.566 | No | Birefringence | 6.77 | N/A | 97.59 |
| 7 day | | | form | | | | |
| | | | change | | | | |

### Example 3: Scaled-Up Nanosuspension Preparation for Form H^{A} (475 g Scale)

### Preparation

A nanosuspension formulation resembling Formulation 2A, with a vehicle formulation of PVP-VA64/SDS/water=3/0.1/86.9 was prepared at a 475 g scale by dynomill at a target Compound 1 concentration of 100 mg/mL. The following procedure was used. Vehicle formulation (PVP-VA64/SDS/water=3/0.1/86.9 by weight) was added to a glass vial and dissolved using magnetic stirring and sonication to obtain a clear solution. The vehicle solution was mixed with 50 mL of 0.3 mm zirconia beads in an 80 mL grinding chamber. An appropriate amount of Compound 1 Form H^{A} was added into the chamber and the suspension was ground by dynomill. The particle size distribution was monitored during the milling process. When the particle size D₅₀ reached about 150-200 nm, the milling process was halted. The suspension was then diluted with vehicle, as needed, to obtain a homogeneous nanosuspension with the target concentration of 100 mg/mL. Five sub-lots were prepared and combined together to make the final large scale batch. Each sub-lot was characterized independently before combining.

### Characterization

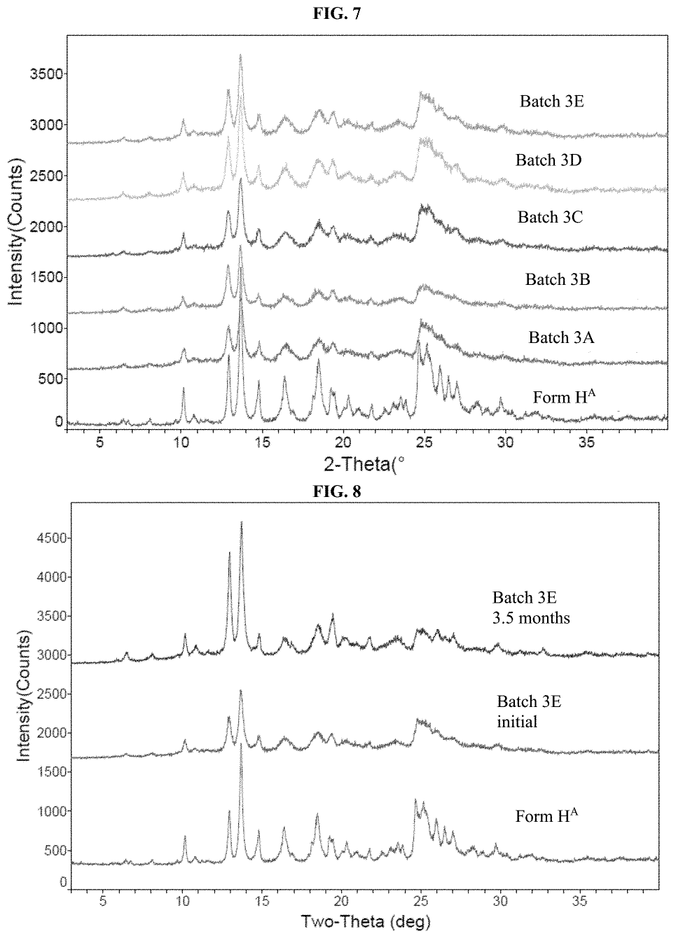

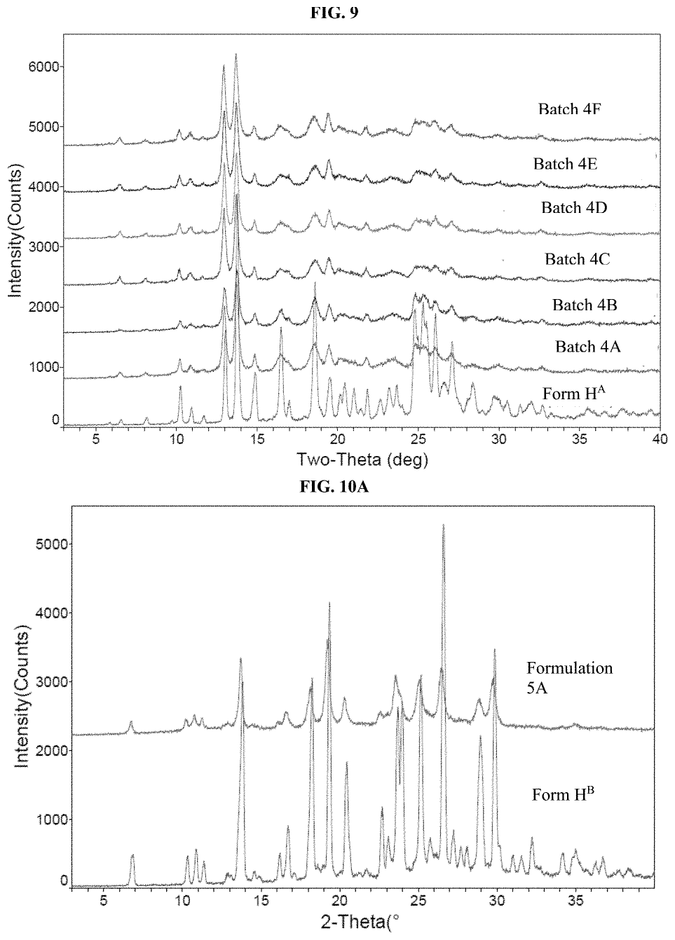

Each sub-lot was characterized by pH measurement, PLM, XRPD and HPLC to determine purity and concentration of Compound 1. The results are shown in Table 9. PLM images and XRPD patterns (FIG. 7) showed that each sub-lot of the nanosuspension contained crystalline Form H^{A} with the same pattern as the starting material, however the crystallinity decreased dramatically. All batches were homogeneous off-white suspensions. Starting material purity was 98.83%, for comparison.

TABLE 9
Characterization of scaled up nanosuspension preparation batches

| | PSD | | | | | HPLC test | | | |
|---|---|---|---|---|---|---|---|---|---|
| | D₅₀ | | | | | Conc. | | Purity | |
| Batch | (nm) | P.I. | XRPD | PLM | pH | (mg/mL) | | (%) | |
| 3A | 167.0 | 0.250 | No form | Birefringence | 5.35 | 1 | 99.5 | 1 | 98.71 |
| | | | change | | | 2 | 96.2 | 2 | 98.68 |
| | | | | | | 3 | 101.5 | 3 | 98.7 |
| | | | | | | Avg | 99.1 | Avg | 98.70 |
| | | | | | | SD | 2.7 | SD | 0.02 |
| 3B | 168.5 | 0.187 | No form | Birefringence | 5.70 | 1 | 101.3 | 1 | 98.98 |
| | | | change | | | 2 | 99.5 | 2 | 98.83 |
| | | | | | | 3 | 100.9 | 3 | 98.97 |
| | | | | | | Avg | 100.6 | Avg | 98.93 |
| | | | | | | SD | 0.9 | SD | 0.08 |
| 3C | 160.6 | 0.233 | No form | Birefringence | 5.61 | 1 | 101.2 | 1 | 98.97 |
| | | | change | | | 2 | 101.5 | 2 | 98.88 |
| | | | | | | 3 | 101.7 | 3 | 98.97 |
| | | | | | | Avg | 101.5 | Avg | 98.94 |
| | | | | | | SD | 0.2 | SD | 0.05 |
| 3D | 168.8 | 0.175 | No form | Birefringence | 5.68 | 1 | 103.2 | 1 | 98.89 |
| | | | change | | | 2 | 102.4 | 2 | 98.95 |
| | | | | | | 3 | 103.0 | 3 | 98.96 |
| | | | | | | Avg | 102.9 | Avg | 98.93 |
| | | | | | | SD | 0.4 | SD | 0.04 |
| 3E | 164.8 | 0.174 | No form | Birefringence | 5.43 | 1 | 104.5 | 1 | 98.97 |
| | | | change | | | 2 | 105.1 | 2 | 98.92 |
| | | | | | | 3 | 105.2 | 3 | 98.91 |
| | | | | | | Avg | 104.9 | Avg | 98.93 |
| | | | | | | SD | 0.4 | SD | 0.03 |

### 3.5 Month Stability of Nanosuspension

About 200 mL of batch 3E was stored in a 4° C. freezer (sealed and protect from light). After about 3.5 months, chemical and physical stability of the batch 3E sample was studied. The results are summarized in Table 10. PLM and XRPD patterns indicated that batch 3E remained crystalline after 3.5 months at 4° C., with the same pattern as starting material. The particle size distribution results showed a D₅₀ of about 125 nm with minimal change from the original suspension. The Compound 1 purity was almost the same as the starting material. Both before and after the 3.5 month storage, the sample was a homogeneous off-white suspension.

TABLE 10
Characterization of Batch 3E after 3.5 months at 4° C.

| | PSD | | | | | HPLC test | | | |
|---|---|---|---|---|---|---|---|---|---|
| | D₅₀ | | | | | Assay | | Purity | |
| Conditions | (nm) | P.I. | XRPD | PLM | pH | (mg/mL) | | (%) | |
| Initial | 164.8 | 0.174 | No form | Birefringence | 5.43 | 1 | 104.5 | 1 | 98.97 |
| | | | change | | | 2 | 105.1 | 2 | 98.92 |
| | | | | | | 3 | 105.2 | 3 | 98.91 |
| | | | | | | Avg. | 104.9 | Avg. | 98.93 |
| | | | | | | SD | 0.4 | SD | 0.03 |

| | | | | | | | |
|---|---|---|---|---|---|---|---|
| 4° C., sealed, | 123.5 | 0.146 | No form | Birefringence | N/A | N/A | 98.84 |
| 3.5 M | | | change | | | | |

### Example 4: Scaled-Up Nanosuspension Preparation for Form H^{A} (1400 g Scale)

### Preparation

A nanosuspension formulation resembling Formulation 2A, with a vehicle formulation of PVP-VA64/SDS/water=3/0.1/86.9 was prepared at a 1400 g scale by dynomill at a target Compound 1 concentration of 100 mg/mL. The following procedure was used. Vehicle formulation (PVP-VA64/SDS/water=3/0.1/86.9 by weight) was added to a glass vial and dissolved using magnetic stirring (700 rpm) and sonication to obtain a clear solution. The vehicle solution was mixed with 50 mL of 0.3 mm zirconia beads in an 80 mL grinding chamber. An appropriate amount of Compound 1 Form H^{A} was added into the chamber and the suspension was ground by dynomill. The particle size distribution was monitored during the milling process. When the particle size D₅₀ reached about 150-200 nm, the milling process was halted. The suspension was then diluted with vehicle, as needed, to obtain a homogeneous nanosuspension with the target concentration of 100 mg/mL. Six sub-lots were prepared and combined together to make the final large scale batch. Each sub-lot was characterized independently before combining.

### Characterization

Each sub-lot was characterized by pH measurement, PLM, XRPD, PLC to determine purity and UPLC to determine concentration of Compound 1. The results are shown in Table 11. PLM images and XRPD patterns showed that each sub-lot of the nanosuspension contained crystalline Form H^{A} with the same pattern as the starting material, however the crystallinity decreased dramatically. All batches were homogeneous off-white suspensions. The particle size distribution results showed D₅₀ was about 160-200 nm with a variance lower than 0.3 for each sub-lot sample. HPLC results showed that Compound 1 concentrations were within 20% of the target concentration of 100 mg/mL for each sub-lot. The Compound 1 purity was determined to be 100% for each sub-lot sample.

TABLE 11
Characterization of scaled up nanosuspension preparation batches

| | | PSD | | | | | | HPLC test | | |
|---|---|---|---|---|---|---|---|---|---|---|
| | Volume | D₅₀ | D₉₀ | | | | | Assay | | Purity |
| Batch | (mL) | (nm) | (nm) | P.I. | XRPD | PLM | pH | (mg/mL) | | (%) |
| 4A | 800 | 191.7 | 350.6 | 0.222 | No form | Birefringence | 4.96 | 1 | 102.0 | 100 |
| | | | | | change | | | 2 | 101.1 | |
| | | | | | | | | 3 | 102.1 | |
| | | | | | | | | Avg. | 101.7 | |
| | | | | | | | | SD | 0.6 | |
| 4B | 5200 | 175.1 | 326.3 | 0.236 | No form | Birefringence | 5.06 | 1 | 99.4 | 100 |
| | | | | | change | | | 2 | 97.7 | |
| | | | | | | | | 3 | 99.7 | |
| | | | | | | | | 4 | 99.4 | |
| | | | | | | | | 5 | 99.9 | |
| | | | | | | | | Avg. | 99.2 | |
| | | | | | | | | SD | 0.86 | |
| 4C | 2250 | 195.8 | 315.4 | 0.138 | No form | Birefringence | 5.04 | 1 | 102.2 | 100 |
| | | | | | change | | | 2 | 99.6 | |
| | | | | | | | | 3 | 100.9 | |
| | | | | | | | | 4 | 100.7 | |
| | | | | | | | | 5 | 100.8 | |
| | | | | | | | | Avg. | 100.8 | |
| | | | | | | | | SD | 0.93 | |
| 4D | 2600 | 185.0 | 324.7 | 0.193 | No form | Birefringence | 5.05 | 1 | 100.9 | 100 |
| | | | | | change | | | 2 | 100.7 | |
| | | | | | | | | 3 | 100.1 | |
| | | | | | | | | 4 | 98.3 | |
| | | | | | | | | 5 | 99.2 | |
| | | | | | | | | Avg. | 99.8 | |
| | | | | | | | | SD | 1.09 | |
| 4E | 1965 | 172.4 | 298.8 | 0.184 | No form | Birefringence | 5.07 | 1 | 99.2 | 100 |
| | | | | | change | | | 2 | 98.8 | |
| | | | | | | | | 3 | 101.6 | |
| | | | | | | | | 4 | 100.4 | |
| | | | | | | | | 5 | 101.1 | |
| | | | | | | | | Avg. | 100.2 | |
| | | | | | | | | SD | 1.18 | |
| 4F | 260 | 160.9 | 276.6 | 0.179 | No form | Birefringence | 5.07 | 1 | 101.4 | 100 |
| | | | | | change | | | | | |

### Example 5: Nanosuspensions Comprising Additional Forms

### Preparation

Nanosuspension formulations comprising Compound 1 Forms H^{B} and D were prepared, using a vehicle formulation of PVP-VA64/SDS/water=3/0.1/86.9 and a target Compound 1 concentration of 100 mg/mL. The following procedure was used for both Forms. Vehicle formulation (PVP-VA64/SDS/water=3/0.1/86.9 by weight) was added to a glass vial and dissolved using magnetic stirring and sonication to obtain a clear solution. The vehicle solution was mixed with 0.3 mm zirconia beads at a ratio of 3:1 (v/v) in a 125 mL grinding chamber. An appropriate amount of Compound 1 Form H^{B} or D was added into the chamber and the suspension was ground by planetary ball mill (PM400) at 300 rpm. The particle size distribution was monitored during the milling process. When the particle size D₅₀ reached about 300 nm or the particle size had no obvious change over time, the milling process was halted. The suspension was then diluted with vehicle, as needed, to obtain a homogeneous nanosuspension with the target concentration of 100 mg/mL.

TABLE 12
Form H^{B} and Form D nanosuspensions

| Formulation | 5A | 5B |
|---|---|---|
| Form | Free base | Free base |
| | hydrate H_{B} | anhydrate D |
| Composition | API/PVP-VA64/ | API/PVP-VA64/ |
| (by weight) | SDS/water = | SDS/water = |
| | 10/3/0.1/86.9 | 10/3/0.1/86.9 |
| Volume of | 45 | 37.5 |
| vehicle | | |
| Milling time | 1.25 | 1.25 |
| (h) | | |
| Last D₅₀ (nm) | 234.2 | 243.5 |

### Characterization

The nanosuspensions were characterized by PLM, XRPD and HPLC to determine purity and concentration of Compound 1. The results are shown in Table 13.

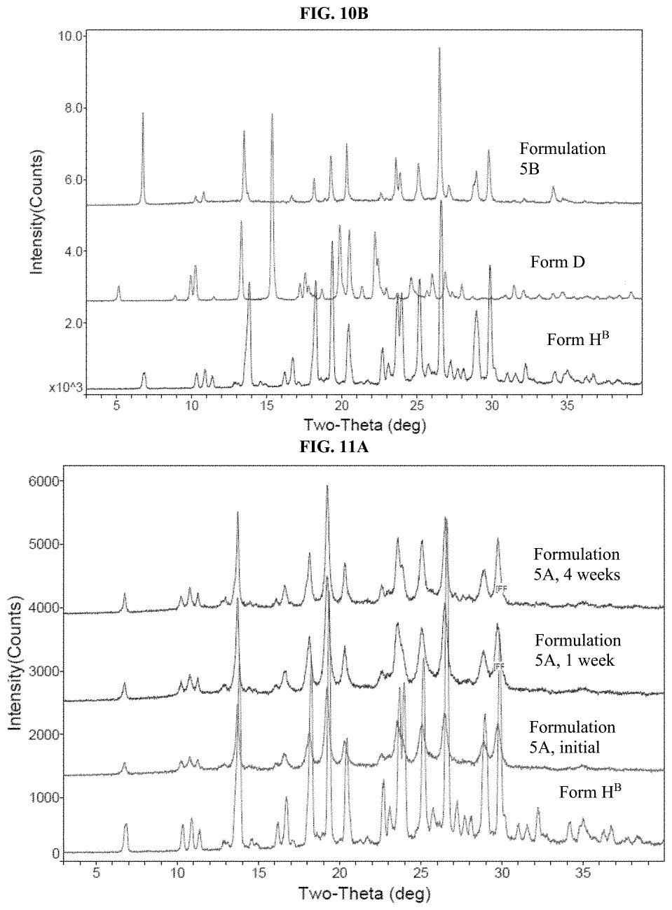

PLM images and XRPD patterns (FIG. 10A) showed that Formulation 5A contained crystalline Form H^{B} with the same pattern as the starting material, however the crystallinity decreased dramatically. The sample was a homogeneous off-white suspension. The particle size distribution analysis showed a D₅₀ of about 240 nm with a variance lower than 0.3. HPLC results showed a concentration of Compound 1 of 99.7 mg/mL and the same Compound 1 purity as the starting material (starting materials purity was 98.83%).

PLM images and XRPD patterns (FIG. 10B) showed that Formulation 5B contained crystalline material, but the XRPD peaks more closely resembled those for Form H^{B} based on characteristic peaks. The sample was a homogeneous off-white suspension. The particle size distribution analysis showed a D₅₀ of about 2500 nm, with a variance of about 1. HPLC results showed a concentration of Compound 1 of 101.3 mg/mL and the same Compound 1 purity as the starting material (starting material purity was 99.7%).

TABLE 13
Characterization of Form H^{B} and Form D nanosuspensions

| | PSD | | | | | HPLC test | | | |
|---|---|---|---|---|---|---|---|---|---|
| | D₅₀ | | | | | Conc. | | Purity | |
| Formula | (nm) | P.I. | XRPD | PLM | pH | (mg/mL) | | (%) | |
| 5A | 234.6 | 0.218 | No | Birefringence | 5.70 | 1 | 99.1 | 1 | 98.77 |
| | | | form | | | 2 | 100.4 | 2 | 98.69 |
| | | | change | | | 3 | 99.5 | 3 | 98.69 |
| | | | | | | Avg. | 99.7 | N/A | N/A |
| | | | | | | SD | 0.7 | N/A | N/A |
| 5B | Initial | Initial | Change | Birefringence | 5.19 | 1 | 102.2 | 1 | 99.70 |
| | 2551.6 | 1.040 | to be | | | 2 | 99.8 | 2 | 99.71 |
| | 2 Days | 2 Days | similar | | | 3 | 102.0 | 3 | 99.67 |
| | 2511.9 | 0.949 | with | | | Avg. | 101.3 | N/A | N/A |
| | | | hydrate | | | SD | 1.3 | N/A | N/A |
| | | | H_{B} | | | | | | |

### 1-Week and 4-Week Stability Studies on Formulation 5A and 5B

The chemical and physical stability of Formulation 5A and 5B nanosuspensions were studied under 25° C./60% RH (sealed and protected from light) conditions. The nanosuspensions were transferred into 4-mL glass vials with locking lids. The vials were covered by aluminum foil and stored in a stability chamber at 25° C./60% RH for 1 week or 4 weeks. The 1-week and 4-week stability study results for Formulations 5A and 5B are reported below in Tables 14A and 14B.

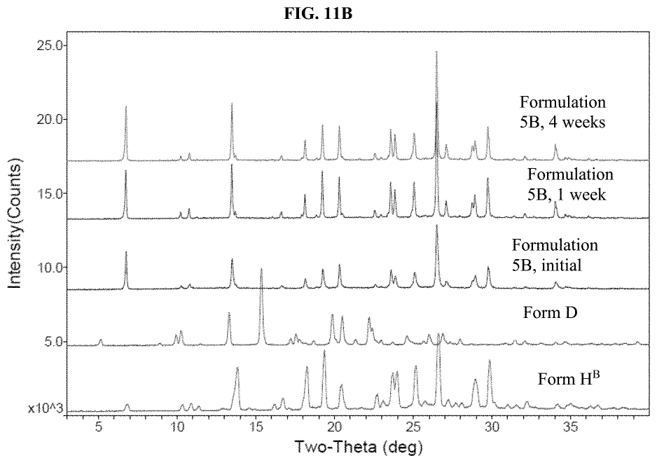

PLM and XRPD patterns (FIG. 11A) for Formulation 5A after both 1 week and 4 weeks showed that the Compound 1 material remained crystalline with the same pattern as the starting material. The particle size distribution results showed that D₅₀ increased to ˜300 nm at 1 week and ˜285 at 4 weeks. The purity HPLC results showed that the Compound 1 in Formulation 5A was stable and the purity remained almost the same as starting material over both the 1 week study and the 4 week study. The appearance of all materials remained a homogeneous tan suspension.

TABLE 14A
1 week and 4 week stability data for Formulation 5A

| | PSD | | | | | HPLC | | | |
|---|---|---|---|---|---|---|---|---|---|
| | D₅₀ | Variance | | | | Conc. | | Purity | |
| Conditions | (nm) | (P.I.) | XRPD | PLM | pH | (mg/mL) | | (%) | |
| Initial | 234.6 | 0.218 | No form | Birefringence | 5.70 | 1 | 99.1 | 1 | 98.77 |
| | | | change | | | 2 | 100.4 | 2 | 98.69 |
| | | | | | | 3 | 99.5 | 3 | 98.69 |

| 25° C./60% RH, | 300.6 | 0.136 | No form | Birefringence | 5.39 | N/A | 98.84 |
|---|---|---|---|---|---|---|---|
| closed, 1 W | | | change | | | | |
| 25° C./60% RH, | 286.5 | 0.136 | No form | Birefringence | 5.79 | N/A | 98.89 |

| | |
|---|---|
| closed, 4 W | change |

PLM and XRPD patterns (FIG. 11B) for Formulation 5B after both 1 week and 4 weeks showed that the Compound 1 material was crystalline and retained characteristic XRPD peaks that matched Form H^{B}. The particle size distribution results showed that D₅₀ remained at ˜2500 after 1 week and 4 weeks. The purity HPLC results showed that the Compound 1 in Formulation 5B was stable and the purity remained almost the same as starting material over both the 1 week study and the 4 week study. After both 1 week and 4 weeks, the suspensions became inhomogeneous, having two layers.

TABLE 14B
1 week and 4 week stability data for Formulation 5B

| | PSD | | | | | HPLC | | | |
|---|---|---|---|---|---|---|---|---|---|
| | D₅₀ | Variance | | | | Conc. | | Purity | |
| Conditions | (nm) | (P.I.) | XRPD | PLM | pH | (mg/mL) | | (%) | |
| Initial | 2551.6 | 1.040 | Similar | Birefringence | 5.19 | 1 | 102.2 | 1 | 99.70 |
| | | | with | | | 2 | 99.8 | 2 | 99.71 |
| | | | hydrate | | | 3 | 102.0 | 3 | 99.67 |

| | | | | | | | |
|---|---|---|---|---|---|---|---|
| | | | H^{B} | | | | |
| 25° C./60% RH, | 2291.5 | 0.760 | Similar | Birefringence | 5.38 | N/A | 99.71 |
| closed, 1 W | | | with | | | | |
| | | | hydrate | | | | |
| | | | H^{B} | | | | |
| 25° C./60% RH, | 2505.4 | 1.263 | Similar | Birefringence | 5.43 | N/A | 99.68 |
| closed, 4 W | | | with | | | | |
| | | | hydrate | | | | |
| | | | H^{B} | | | | |

### Example 6: In Vivo Pharmacokinetic Data in Dogs

Animal Care

Fresh drinking water was available to all subjects, ad libitum. Subjects were fed twice daily. For PO dose groups, subjects were fed the afternoon (3:30-4:00 pm) prior to the day of dosing and the remaining food was removed at about 7:00 pm. Food was withheld until 4-hours post-dose.

Formulation

For studies using 100 mg/kg dosage, the target dose concentration for each administration was 20 mg/mL with a target dose volume of 5 mL/kg. The 100 mg/kg dose nanosuspension formulations were prepared for administration by mixing 1 mL of the nanosuspension (100 mg/mL initial concentration, as prepared in the preceding examples) with 4 mL of water to create the dosing solution of 20 mg/mL of API. For studies using 180 mg/kg dosage, the target dosage concentration for each administration was 36 mg/mL with a target dose volume of 5 mL/kg. The 180 mg/kg dose nanosuspension formulation was prepared for administration by mixing 54 mL of the nanosuspension (100 mg/mL initial concentration, as prepared in the preceding examples) with 96 mL of water to create the dosing solution of 36 mg/mL of API. For studies using 10 mg/kg dosage, the target dosage concentration for each administration was 2 mg/mL with a target dose volume of 5 mL/kg. The 10 mg/kg dose nanosuspension formulation was prepared for administration by mixing 3 mL of the nanosuspension (100 mg/mL initial concentration, as prepared in the preceding examples) with 147 mL of water to create the dosing solution of 2 mg/mL of API.

The concentration of Compound 1 in each formulation was confirmed by UPLC by collecting aliquots from the bottom, middle and top regions of the dosing solutions. All formulation samples were stored at ˜2-8° C. until analyzed.

Administration

Subjects were fasted overnight through approximately 2-4 hours post-dosage. Subjects were weighted prior to dose administration on each day of dosing to calculate the actual dose volume. Subjects received a single oral gavage administration of the appropriate Formulation.

Blood Collection

Blood samples were collected pre-dose and post dosage at various time points. For example, in certain experiments, blood samples were collected at 0.25, 0.5, 1.0, 2.0, 4.0, 8.0, 16, 24, 30 and 48 hours post dosage. Approximately 1.0 mL of blood was collected at each time point via peripheral vessel from each subject. Blood samples were transferred into tubes containing potassium EDTA (0.85 mg-1.15 mg). Plasma samples were then prepared by centrifuging the blood samples at ˜2-8° C., 3000 g for 10 minutes. All plasma samples were then frozen over dry ice and kept at −60° C. or lower until analysis.

Analysis

The plasma concentration of Compound 1 in each sample was determined by using the LC-MS/MS parameters reported below:

| | |
|---|---|
| Equipment | ACQUITY UPLC System |
| Analytical column | ACQUITY UPLC Protein BEH |
| | C4 300 Å 1.7 μm 2.1 × 50 mm |
| Inject volume | 2 μL |
| Mobile phase A | 2 mM HCOONH₄ in |
| | water:acetonitrile (v:v, 95:5) |
| Mobile phase B | 2 mM HCOONH₄ in |
| | acetonitrile:water (v:v, 95:5) |
| Elution mode | Gradients (see below) |

Gradient 1

| Time (min) | Flow Rate (mL/min) | A % | B % |
|---|---|---|---|
| Initial | 0.65 | 85 | 15 |
| 1.2 | 0.65 | 5 | 95 |
| 1.4 | 0.65 | 5 | 95 |
| 1.41 | 0.65 | 85 | 15 |
| 1.6 | 0.65 | 85 | 15 |

Gradient 2

| Time (min) | Flow Rate (mL/min) | A % | B % |
|---|---|---|---|
| Initial | 0.65 | 80 | 20 |
| 0.8 | 0.65 | 65 | 35 |
| 1.2 | 0.65 | 5 | 95 |
| 1.4 | 0.65 | 5 | 95 |
| 1.41 | 0.65 | 80 | 20 |
| 1.6 | 0.65 | 80 | 20 |

| | |
|---|---|
| Mass spectrometer | Triple Quad 6500 Plus |
| Ionization mode | ESI(+) |
| Detective mode | MRM |

Plasma concentration data was subjected to a non-compartmental pharmacokinetic analysis using the Phoenix WinNonlin software (version 6.3, Pharsight, Mountain View, Calif.). The linear/log trapezoidal rule was applied in obtaining the PK parameters. Individual plasma concentration values that were below the lower limit of quantitation were excluded from the PK parameter calculation. All plasma concentrations and pharmacokinetic parameters were reported with three significant figures. The nominal dose levels and nominal sampling times were used in the calculation of all pharmacokinetic parameters.

### Formulations 1A, 1B, 1C and 1D

Three (3) non-naive male beagle dogs each were dosed with Formulations 1A, 1B, 1C and 1D (described in Example 1 herein) at 100 mg/kg, by single oral administration in separate phases of a four-phase experiment. Between each phase, there was a 3-day washout period to allow for clearance of Compound 1 from the test subjects. The only clinical observation of note during the experiment was a small amount of liquid feces in one subject administered Formulation 1B, 8 hours post-dose. Otherwise, no adverse side effects were observed.

Results

TABLE 15A
Mean plasma concentrations (ng/mL) of
Compound 1 in male beagle dogs following oral
administration of Formulation 1A at 100 mg/kg

| Time (h) | Mean | SD | CV (%) |
|---|---|---|---|
| 0.0 | — | — | — |
| 0.25 | 1390 | 225 | 16.2 |
| 0.5 | 3043 | 882 | 29.0 |
| 1 | 4320 | 1834 | 42.5 |
| 2 | 4603 | 1705 | 37.0 |
| 4 | 4243 | 1577 | 37.2 |
| 8 | 3203 | 1337 | 41.7 |
| 24 | 3987 | 1462 | 36.7 |
| 30 | 2330 | 875 | 37.5 |
| 48 | 738 | 233 | 31.6 |

TABLE 15B
Mean plasma concentrations (ng/mL) of Compound 1
in male beagle dogs following oral administration of
Formulation 1B at 100 mg/kg

| Time (h) | Mean | SD | CV (%) |
|---|---|---|---|
| 0.0 | 25.6 | 21.8 | 85.2 |
| 0.25 | 540 | 719 | 133 |
| 0.5 | 1720 | 606 | 35.3 |
| 1 | 3367 | 772 | 22.9 |
| 2 | 4693 | 1657 | 35.3 |
| 4 | 4820 | 1811 | 37.6 |
| 8 | 4163 | 2377 | 57.1 |
| 24 | 2940 | 1863 | 63.4 |
| 30 | 1767 | 1867 | 106 |
| 48 | 1149 | 1915 | 167 |

TABLE 15C
Mean plasma concentrations (ng/mL) of Compound 1
in male beagle dogs following oral administration of
Formulation 1C at 100 mg/kg

| Time (h) | Mean | SD | CV (%) |
|---|---|---|---|
| 0.0 | 6.35 | 5.45 | 85.9 |
| 0.25 | 919 | 673 | 73.2 |
| 0.5 | 2813 | 840 | 29.9 |
| 1 | 4307 | 1148 | 26.7 |
| 2 | 5530 | 2058 | 37.2 |
| 4 | 5817 | 1405 | 24.2 |
| 8 | 4473 | 1260 | 26.4 |
| 24 | 5127 | 1131 | 22.1 |
| 30 | 3013 | 918 | 30.5 |
| 48 | 1403 | 821 | 58.5 |

TABLE 15D
Mean plasma concentrations (ng/mL) of Compound 1
in male beagle dogs following oral administration of
Formulation 1D at 100 mg/kg

| Time (h) | Mean | SD | CV (%) |
|---|---|---|---|
| 0.0 | 111 | 160 | 143 |
| 0.25 | 1827 | 611 | 33.4 |
| 0.5 | 3387 | 346 | 10.2 |
| 1 | 4100 | 658 | 16.1 |
| 2 | 5207 | 1213 | 23.3 |
| 4 | 4630 | 1387 | 30.0 |
| 8 | 3963 | 1347 | 34.0 |
| 24 | 4093 | 1465 | 35.8 |
| 30 | 2687 | 1044 | 38.8 |
| 48 | 1304 | 661 | 50.7 |

TABLE 15E
Mean pharmacokinetic parameters of Compound 1
in male beagle dogs. Values reported as mean
(std dev).

| Formulation | 1A | 1B | 1C | 1D |
|---|---|---|---|---|
| Cₘₐₓ (ng/mL) | 5317 | 5040 | 6587 | 5833 |
| | (934) | (1802) | (1054) | (150) |
| Tₘₐₓ (h) | 16.7 | 4.67 | 10.0 | 9.33 |
| | (12.7) | (3.06) | (12.2) | (12.7) |
| T_{1/2} (h) | 10.8 | 18.1 | 14.5 | 15.0 |
| | (1.48) | (23.7) | (6.34) | (ND) |
| AUC₀₋ₗₐₛₜ | 131044 | 127522 | 180044 | 152602 |
| (h · ng/mL) | (17412) | (91822) | (25713) | (13584) |
| AUC_{0-inf} | 142198 | 201269 | 213303 | 167841 |
| (h · ng/mL) | (17735) | (218429) | (49741) | (ND) |

Formulation 2A after Freeze Thaw

Three (3) non-naive male beagle dogs each were dosed with Formulation 2A after the freeze/thaw study described in Example 2 at 100 mg/kg, by single oral administration as part of a three-phase experiment with two other Compound 1 formulations. Between each phase, there was a 3-day washout period to allow for clearance of Compound 1 from the test subjects. No adverse side effects were observed in the subjects after administration of Formulation 2A.

Results

TABLE 16A
Mean plasma concentrations (ng/mL) of Compound 1
in male beagle dogs following oral administration of
Formulation 2A at 100 mg/kg

| Time (h) | Mean | SD | CV (%) |
|---|---|---|---|
| 0.0 | — | — | — |
| 0.25 | 934 | 413 | 44.2 |
| 0.5 | 2783 | 440 | 15.8 |
| 1 | 3263 | 680 | 20.8 |
| 2 | 4087 | 821 | 20.1 |
| 4 | 4173 | 595 | 14.3 |
| 8 | 4187 | 2485 | 59.3 |
| 16 | 5127 | 2357 | 46.0 |
| 24 | 2867 | 1668 | 58.2 |
| 30 | 1215 | 1018 | 83.8 |
| 48 | 15.3 | 12.8 | 84.1 |

TABLE 16B
Mean pharmacokinetic parameters of Compound 1
in male beagle dogs. Values reported as mean (std dev).

| Formulation | 2A |
|---|---|
| Cₘₐₓ (ng/mL) | 5843 |
| | (1731) |
| Tₘₐₓ (h) | 9.33 |
| | (6.11) |
| T_{1/2} (h) | 3.07 |
| | (0.151) |
| AUC₀₋ₗₐₛₜ (h · ng/mL) | 114951 |
| | (49031) |
| AUC_{0-inf} (h · ng/mL) | 115020 |
| | (49089) |

### Formulations 5A and 5B

Three (3) non-naive male beagle dogs each were dosed with Formulations 5A and 5B (described in Example 5 herein) at 100 mg/kg, by single oral administration as part of a three-phase experiment with one other Compound 1 formulation. Between each phase, there was a 3-day washout period to allow for clearance of Compound 1 from the test subjects. No adverse side effects were observed in the subjects after administration of either Formulation 5A or 5B.

Results

TABLE 17A
Mean plasma concentrations (ng/mL) of Compound 1
in male beagle dogs following oral administration of
Formulation 5A at 100 mg/kg

| Time (h) | Mean | SD | CV (%) |
|---|---|---|---|
| 0.0 | — | — | — |
| 0.25 | 569 | 116 | 20.3 |
| 0.5 | 1373 | 332 | 24.2 |
| 1 | 2477 | 396 | 16.0 |
| 2 | 3133 | 546 | 17.4 |
| 4 | 3440 | 702 | 20.4 |
| 8 | 2950 | 826 | 28.0 |
| 12 | 2387 | 775 | 32.5 |
| 12.25 | 2263 | 781 | 34.5 |
| 12.5 | 2320 | 655 | 28.2 |
| 13 | 2237 | 676 | 30.2 |
| 14 | 2113 | 765 | 36.2 |
| 16 | 1750 | 720 | 41.1 |
| 20 | 1418 | 708 | 49.9 |
| 24 | 1214 | 775 | 63.8 |
| 30 | 692 | 573 | 82.8 |
| 48 | 116 | 113 | 97.5 |

TABLE 17B
Mean plasma concentrations (ng/mL) of Compound 1
in male beagle dogs following oral administration of
Formulation 5B at 100 mg/kg

| Time (h) | Mean | SD | CV (%) |
|---|---|---|---|
| 0.0 | — | — | — |
| 0.25 | 655 | 682 | 104 |
| 0.5 | 1485 | 1058 | 71.3 |
| 1 | 1960 | 1095 | 55.9 |
| 2 | 2703 | 1087 | 40.2 |
| 4 | 1893 | 722 | 38.1 |
| 8 | 2619 | 2195 | 83.8 |
| 12 | 4877 | 386 | 7.91 |
| 12.25 | 5040 | 364 | 7.22 |
| 12.5 | 4957 | 336 | 6.78 |
| 13 | 5307 | 1161 | 21.9 |
| 14 | 5167 | 760 | 14.7 |
| 16 | 4963 | 932 | 18.8 |
| 20 | 4043 | 304 | 7.51 |
| 24 | 3477 | 718 | 20.7 |
| 30 | 2463 | 725 | 29.4 |
| 48 | 999 | 687 | 68.8 |

TABLE 17C
Mean pharmacokinetic parameters of Compound 1 in
male beagle dogs. Values reported as mean (std dev).

| Formulation | 5A | 5B |
|---|---|---|
| Cₘₐₓ (ng/mL) | 3340 | 5657 |
| | (702) | (621) |
| Tₘₐₓ (h) | 4.00 | 11.3 |
| | (0) | (2.89) |
| T_{1/2} (h) | 6.27 | 13.1 |
| | (2.84) | (5.81) |
| AUC₀₋ₗₐₛₜ (h · ng/mL) | 65120 | 131803 |
| | (23758) | (18281) |
| AUC_{0-inf} (h · ng/mL) | 66477 | 154421 |
| | (24285) | (33493) |

Formulation 4A at 180 mg/kg or 10 mg/kg

Three (3) non-naive male beagle dogs each were dosed with Formulation 4A (described in Example 4 herein) at 180 mg/kg or 10 mg/kg, by single oral administration as part of a nine-phase experiment with seven other Compound 1 formulations and/or dosages. Between each phase, there was a 3-day washout period to allow for clearance of Compound 1 from the test subjects. One of the subjects administered Formulation 4A at 180 mg/kg produced a small amount of vomit at 20 h post dosage. Otherwise, no adverse side effects were observed in the subjects after administration of either dosage of Formulation 4A.

Results

TABLE 18A
Mean plasma concentrations (ng/mL) of Compound 1
in male beagle dogs following oral administration of
Formulation 4A at 180 mg/kg

| Time (h) | Mean | SD | CV (%) |
|---|---|---|---|
| 0.0 | — | — | — |
| 0.25 | 321 | 180 | 56.2 |
| 0.5 | 1276 | 244 | 19.1 |
| 1 | 2263 | 445 | 19.7 |
| 2 | 2917 | 270 | 9.27 |
| 4 | 3377 | 855 | 25.3 |
| 8 | 2753 | 584 | 21.2 |
| 12 | 2083 | 621 | 29.8 |
| 16 | 2217 | 1737 | 78.4 |
| 20 | 2740 | 2935 | 107 |
| 24 | 2345 | 2873 | 123 |
| 30 | 1737 | 2245 | 129 |
| 48 | 694 | 966 | 139 |

TABLE 18B
Mean plasma concentrations (ng/mL) of
Compound 1 in male beagle dogs following oral
administration of Formulation 4A at 10 mg/kg

| Time (h) | Mean | SD | CV (%) |
|---|---|---|---|
| 0.0 | — | — | — |
| 0.25 | 332 | 362 | 109 |
| 0.5 | 1747 | 982 | 56.2 |
| 1 | 2643 | 1156 | 43.7 |
| 2 | 2867 | 830 | 28.9 |
| 4 | 2117 | 874 | 41.3 |
| 8 | 1218 | 523 | 43.0 |
| 12 | 542 | 374 | 69.0 |
| 16 | 294 | 267 | 91.1 |
| 20 | 173 | 210 | 121 |
| 24 | 80.4 | 120 | 150 |
| 30 | 19.1 | — | — |
| 48 | — | — | — |

TABLE 18C
Mean pharmacokinetic parameters of Compound 1,
Formulation 4A in male beagle dogs. Values reported
as mean (std dev).

| Formulation | 180 mg/kg | 10 mg/kg |
|---|---|---|
| Cₘₐₓ (ng/mL) | 3963 | 3023 |
| | (1869) | (983) |
| Tₘₐₓ (h) | 9.33 | 1.67 |
| | (9.24) | (0.577) |
| T_{1/2} (h) | 10.8 | 2.14 |
| | (5.17) | (0.586) |
| AUC₀₋ₗₐₛₜ (h · ng/mL) | 92974 | 22090 |
| | (74810) | (9070) |
| AUC_{0-inf} (h · ng/mL) | 107211 | 22144 |
| | (95184) | (9135) |

Those skilled in the art will recognize, or be able to ascertain, using no more than routine experimentation, numerous equivalents to the specific compositions and procedures described herein. Such equivalents are considered to be within the scope of this disclosure, and are covered by the following claims.

What is claimed is:

1. A pharmaceutical composition comprising:
(i) N-(5-(5-((1R,2S)-2-fluorocyclopropyl)-1,2,4-oxadiazol-3-yl)-2-methylphenyl)imidazo[1,2-a]pyridine-3-carboxamide in an amount of about 5 wt % to about 15 wt %;
(ii) a stabilizer in an amount of about 1 wt % to about 10 wt %;
(iii) a surfactant in an amount of about 0.05 wt % to about 0.15 wt %; and
(iv) water.

2. The pharmaceutical composition of claim 1, wherein the pharmaceutical composition is a nanosuspension comprising nanoparticles of N-(5-(5-((1R,2S)-2-fluorocyclopropyl)-1,2,4-oxadiazol-3-yl)-2-methylphenyl)imidazo[1,2-a]pyridine-3-carboxamide suspended in an aqueous solution.

3. The pharmaceutical composition of claim 1, wherein the N-(5-(5-((1R,2S)-2-fluorocyclopropyl)-1,2,4-oxadiazol-3-yl)-2-methylphenyl)imidazo[1,2-a]pyridine-3-carboxamide is in the form of nanoparticles comprising free base Form H^{A} of N-(5-(5-((1R,2S)-2-fluorocyclopropyl)-1,2,4-oxadiazol-3-yl)-2-methylphenyl)imidazo[1,2-a]pyridine-3-carboxamide.

4. The pharmaceutical composition of claim 3, wherein free base Form H^{A} of N-(5-(5-((1R,2S)-2-fluorocyclopropyl)-1,2,4-oxadiazol-3-yl)-2-methylphenyl)imidazo[1,2-a]pyridine-3-carboxamide is a crystalline solid form having peaks in its X-ray powder diffraction pattern at about 12.8, about 13.6, and about 19.3 degrees 2-theta.

5. The pharmaceutical composition of claim 1, wherein the pharmaceutical composition is a nanosuspension comprising nanoparticles of N-(5-(5-((1R,2S)-2-fluorocyclopropyl)-1,2,4-oxadiazol-3-yl)-2-methylphenyl)imidazo[1,2-a]pyridine-3 -carboxamide having a median particle size of about 100 nm to about 200 nm, with a particle size distribution span less than about 1.5.

6. The pharmaceutical composition of claim 1, wherein the stabilizer is a vinylpyrollidone-vinyl acetate (PVP/VA) copolymer.

7. The pharmaceutical composition of claim 6, wherein the stabilizer is a vinylpyrollidone-vinyl acetate (PVP/VA) copolymer having a weight-average molecular weight of 45,000-70,000 g/mol.

8. The pharmaceutical composition of claim 1, wherein the surfactant is sodium dodecyl sulfate (SDS).

9. The pharmaceutical composition of claim 1, wherein the pharmaceutical composition comprises:
(i) N-(5-(5-((1R,2S)-2-fluorocyclopropyl)-1,2,4-oxadiazol-3-yl)-2-methylphenyl)imidazo[1,2-a]pyridine-3-carboxamide in the form of crystalline free base Form H^{A} nanoparticles having a median particle size of about 100 nm to about 200 nm, with a particle size distribution span less than about 1.5, in an amount of about 8 wt % to about 12 wt %;
(ii) PVP/VA in an amount of about 2 wt % to about 5 wt %;
(iii) SDS in an amount of about 0.08 wt % to about 0.12 wt %; and
(iv) water, wherein the water makes up the mass balance of the composition.

10. The pharmaceutical composition of claim 1, wherein the pharmaceutical composition comprises:
(i) N-(5-(5-((1R,2S)-2-fluorocyclopropyl)-1,2,4-oxadiazol-3-yl)-2-methylphenyl)imidazo[1,2-a]pyridine-3-carboxamide in the form of crystalline free base Form H^{A} nanoparticles having a median particle size of about 100 nm to about 200 nm, with a particle size distribution span less than about 1.5, in an amount of about 10 wt %;
(ii) PVP/VA in an amount of about 3 wt %;
(iii) SDS in an amount of about 0.1 wt %; and
(iv) water, in an amount of about 86.9 wt %.

11. The pharmaceutical composition of claim 1, wherein the N-(5-(5-((1R,2S)-2-fluorocyclopropyl)-1,2,4-oxadiazol-3-yl)-2-methylphenyl)imidazo[1,2-a]pyridine-3-carboxamide is in the form of nanoparticles comprising free base Form H^{B} of N-(5-(5-((1R,2S)-2-fluorocyclopropyl)-1,2,4-oxadiazol-3-yl)-2-methylphenyl)imidazo[1,2-a]pyridine-3-carboxamide.

12. The pharmaceutical composition of claim 11, wherein free base Form H^{B} of N-(5-(5-((1R,2S)-2-fluorocyclopropyl)-1,2,4-oxadiazol-3-yl)-2-methylphenyl)imidazo[1,2-a]pyridine-3-carboxamide is a crystalline solid form having peaks in its X-ray powder diffraction pattern at about 13.6, about 18.0, and about 26.4 degrees 2-theta.

13. The pharmaceutical composition of claim 1, wherein the N-(5-(5-((1R,2S)-2-fluorocyclopropyl)-1,2,4-oxadiazol-3-yl)-2-methylphenyl)imidazo[1,2-a]pyridine-3-carboxamide is in the form of nanoparticles comprising free base Form A of N-(5-(5-((1R,2S)-2-fluorocyclopropyl)-1,2,4-oxadiazol-3-yl)-2-methylphenyl)imidazo[1,2-a]pyridine-3-carboxamide.

14. The pharmaceutical composition of claim 13, wherein free base Form A of N-(5-(5-((1R,2S)-2-fluorocyclopropyl)-1,2,4-oxadiazol-3-yl)-2-methylphenyl)imidazo[1,2-a]pyridine-3-carboxamide is a crystalline solid form having peaks in its X-ray powder diffraction pattern at about 13.2, about 15.2, and about 19.7 degrees 2-theta.

15. A method of inhibiting the activity of a c-kit kinase in a patient, comprising administering to said patient a pharmaceutical composition according to claim 1.

16. A method of treating a c-kit kinase mediated disease or disorder in a patient, comprising administering to said patient a pharmaceutical composition according to claim 1.

17. The method of claim 16, wherein the c-kit kinase mediated disease or disorder is a mast-cell associated disease, a respiratory disease, an inflammatory disorder, an autoimmune disorder, a metabolic disease, a fibrosis disease, or a dermatological disease.

18. The method of claim 16, wherein the c-kit kinase mediated disease or disorder is asthma, allergic rhinitis, pulmonary arterial hypertension (PAH), primary pulmonary hypertension (PPH), pulmonary fibrosis, hepatic fibrosis, cardiac fibrosis, scleroderma, irritable bowel syndrome (IBS), inflammatory bowel disease (IBD), urticaria, dermatosis, atopic dermatitis, allergic contact dermatitis, rheumatoid arthritis, multiple sclerosis, melanoma, a gastrointestinal stromal tumor, a mast cell tumor, mastocytosis, anaphylactic syndrome, food allergy, type I diabetes or type II diabetes.

19. The method of claim 16, wherein the pharmaceutical composition is administered to the patient orally.

20. The method of claim 18, wherein the pharmaceutical composition is administered to the patient orally.
